(19) United States
(12) Patent Application Publication
(10) Pub. No.: US 2005/0053587 A1
(43) Pub. Date: Mar. 10, 2005

(54) Cultured stromal cells and uses thereof

(76) Inventors: Jacques Galipeau, Town of Mount-Royal (CA); Al-Khaldi Abdulaziz, Palo Alto, CA (US); Kevin Lachapelle, Westmount (CA); Nicoletta Eliopoulos, Montreal (CA); John Stagg, Montreal (CA)

Correspondence Address: David S Resnick;Nixon Peabody, 100 Summer Street, Boston, MA, 02110-2131, US

(21) Appl. No.: 10/490,277

(22) PCT Filed: Sep. 19, 2002
(86) PCT No.: PCT/CA02/01435

(51) Int. Cl. A61K 48/00; C12N 5/08

(52) U.S. Cl. 424/93.21; 435/372

(57) ABSTRACT

The present invention relates to genetically-engineered bone marrow stromal cells and method of preparation thereof for ex vivo delivery of protein and peptides of interest into human or animals. The method includes forming a bone marrow stromal cell expression system in vitro and administering the expression system to a human or animal recipient. The invention relates also to implants colonized by bone marrow stromal cells. In accordance with the invention, the implants comprise a matrix which can be composed of a large variety of biocompatible and biodegradable products, and stromal cells which are integrated into the matrix as such or under genetically-engineered forms. Genetically-engineered bone marrow stromal cells or cell colonized implant are also useful for tissue repair and tissue synthesis, as for angiogenesis.

### BACKGROUND OF THE INVENTION

(a) Field of the Invention

The invention relates to genetically-engineered autologous stromal cells for delivery of biologically active protein into a host human or an animal. The invention relates also to the method of preparing the genetically-engineered autologous stromal cells, and implantation of the genetically-engineered cells into a host human or an animal for in vivo delivery of biologically active proteins. Also, the invention relates to implants containing bone marrow stromal cells, which after implantation into a patient, can stimulate or trigger tissue synthesis, tissue repair or modulate the production of different endogenous products, as protein, lipids, glycoproteins, and glucides. The cells of the present invention can be incorporated as under the native form into the implant before implantation, or genetically transformed to be rendered transgenic to secrete proteins of interest.

(b) Description of Prior Art

Gene transfer is now widely recognized as a powerful tool for analysis of biological events and disease processes at both the cellular and molecular level (Murray, E. J., rd. Methods in Molecular Biology, Vol. 7, Humana Press Inc., Clifton, N.J., (1991); Kriegler, M., A Laboratory Manual, W. H. Freeman and Co., New York (1990)). More recently, the application of gene therapy for the treatment of human diseases, either inherited (e.g., ADA deficiency) or acquired (e.g., cancer or infectious disease), has received considerable attention (Mulligan, R. C., Science 260:926-932 (1993), Tolstoshev, P., Annu. Rev. Pharmacol. Toxicol. 32:573-596 (1993), Miller, A. D., Nature 357:455-460 (1992), Anderson, W. F., Science 256:808-813 (1992), and references therein). With the advent of improved gene transfer techniques and the identification of an ever expanding library of “defective gene”-related diseases, gene therapy has rapidly evolved from a treatment theory to a practical reality.

Traditionally, gene therapy has been defined as “a procedure in which an exogenous gene is introduced into the cells of a patient in order to correct an inborn genetic error”. Although more than 4500 human diseases are currently classified as genetic, specific mutations in the human genome have been identified for relatively few of these diseases. Until recently, these rare genetic diseases represented the exclusive targets of gene therapy efforts. Only recently, researchers and clinicians have begun to appreciate that most human cancers, certain forms of cardiovascular disease, and many degenerative diseases also have important genetic components, and for the purposes of designing novel gene therapies, should be considered a “genetic disorders”. Therefore, gene therapy has more recently been broadly defined as “the correction of a disease phenotype through the introduction of new genetic information into the affected organism”.

Two basic approaches to gene therapy have evolved: (1) ex vivo gene therapy and (2) in vivo gene therapy. In ex vivo gene therapy, cells are removed from a subject and cultured in vitro. A functional replacement gene is introduced into the cells (transfection) in vitro, the modified cells are expanded in culture, and then reimplanted in the subject. These genetically modified, reimplanted cells are able to secrete detectable levels of the transfected gene product in situ. The development of improved retroviral gene transfer methods (transduction) has greatly facilitated the transfer into and subsequent expression of genetic material by somatic cells. Accordingly, retrovirus-mediated gene transfer has been used in clinical trials to mark autologous cells and as a way of treating genetic disease.

Systemic transgene delivery has been accomplished by implanting gene-modified autologous cells via intravenous, intramuscular, intraperitoneal, and subcutaneous administration. Cell types explored as gene delivery vehicles encompass skin fibroblasts, myoblasts, vascular smooth muscle cells, hematopoietic stem cells, lymphocytes, and human umbilical vein endothelial cells. However, there are drawbacks associated with the use of these cells in an autologous setting. Skin fibroblasts have been shown to inactivate introduced vector sequences following transplantation and depending on the age of the donor have limited in vitro proliferation capacities, thus requiring the harvest of considerable quantities of primary cells. Skeletal myoblasts are present in very low amounts in the majority of adult mammals, and their successful growth and transplantation is technically challenging . Vascular smooth muscle cells, to engraft in humans, may necessitate arterial injury. Hematopoietic stem cells can be difficult to expand in culture and gene-modify, and very large numbers are required for engraftment in the absence of a toxic “conditioning” regimen. Lymphocytes possess a short lifespan, and human umbilical vein endothelial cells are limited in their use as autologous cells since they cannot be obtained from an adult.

Despite the wide range of cell types tested, a satisfactory target cell for human gene therapy has not yet been identified. The inadequacies of the above-identified cell types include: (1) inefficient or transient expression of the inserted gene; (2) necrosis following subcutaneous injection of cells; (3) limited dissemination of the inserted gene product from the site of transduced cell implantation; and (4) limitations in the amount of therapeutic agent delivered in situ.

In one particular application, it was initially assumed that hematopoietic stem cells would be the primary target cell type used for ex vivo human gene therapy in part, because of the large number of genetic diseases associated with differentiated stem cell lineages. However, because of problems inherent to hematopoietic stem cell transfection (e.g., inefficient transgene expression), more recent gene therapy efforts have been aimed at the identification of alternative cell types for transformation. The cell types that may be included are keratinocytes, fibroblasts, lymphocytes, myoblasts, smooth muscle cells, and endothelial cells.

Implants

A few researchers have explored the use of natural substrates related to basement membrane components. Basement membranes comprise a mixture of glycoproteins and proteoglycans that surround most cells in vivo. For example, collagen has been used for culturing heptocytes, epithelial cells and endothelial tissue. Growth of cells on floating collagen and cellulose nitrate has been used in attempts to promote terminal differentiation. However, prolonged cellular regeneration and the culture of such tissues in such systems have not heretofore been achieved.

While the growth of cells in two dimensions is a convenient method for preparing, observing and studying cells in culture, allowing a high rate of cell proliferation, it lacks the cell-cell and cell-matrix interactions characteristic of whole tissue in vivo.

In general, implant substrates are inoculated with the cells to be cultured. Many of the cell types have been reported to penetrate the matrix and establish a “tissue-like” histology. Various attempts have been made to regenerate tissue-like architecture from dispersed monolayer cultures. Kruse and Miedema (1965, J. Cell Biol. 27:273) reported that perfused monolayers could grow to more than ten cells deep and organoid structures can develop in multilayered cultures if kept supplied with appropriate medium.

However, the long term culture and proliferation of cells in such systems has not been achieved.

Angiogenesis and Ischemic disease

Ischemic Heart Disease (IHD) and peripheral atherosclerotic arterial diseases are major causes of morbidity and mortality in the world. Conventional treatment for both includes minimizing risk factors, medical therapy, and interventional therapies to restore the arterial blood flow either by angioplasty or bypass surgery. It is becoming increasingly evident that there is a growing number of patients suffering from debilitating symptoms who are not candidates for conventional revascularization. There is interest in exploring alternative forms of therapy to ameliorate symptoms and improve blood flow to ischemic tissues for those patients who have run out of therapeutic options.

To provide an adequate treatment to such disease, an ideal implant material would provide a physical support for the cells to keep them evenly dispersed throughout the implant. If cells tend to clump within the implant, the cells in the middle of the clump may be deprived of oxygen and other nutrients and become necrotic. The implant matrix should also be sufficiently permeable to substances secreted by the cells so that a therapeutic substance can diffuse out of the implant and into the tissue or blood stream of the recipient of the implanted vehicle. If proliferation or differentiation of cells within the implant is desired, the implant matrix should also provide a physio-chemical environment which promotes those cellular functions.

A significant drawback in the use of matrices or hydrogels, however, and one that has substantially hindered the use of hydrogels in drug delivery systems is that such formulations are generally not biodegradable. Thus, drug delivery devices formulated with hydrogels typically have to be removed after subcutaneous or intramuscular application or cannot be used at all if direct introduction into the blood stream is necessary. Thus, it would be advantageous to use implant that could be degraded after application in the body without causing toxic or other adverse reactions.

There is a great need for methodologies to enhance engraftment of cells in a host animal, and particularly mammals, for the purpose of improved human cell transplantation therapy as well as for improved ex vivo gene therapy. It would be desirable to develop retroviral vectors that integrate into the genome, express desired levels of the gene product of interest, and are produced in high titers with the co-production or expression of marker products such as cytidine deaminase drug resistance.

It would be highly desirable to be provided with a biocompatible and biodegradable implant allowing physiologically the regeneration, repair and stimulation of tissues in a patient in needs.

### SUMMARY OF THE INVENTION

One object of the present invention is to provide an isolated transgenic bone marrow stromal cell for in vivo delivery of a protein of interest into a patient, wherein the stromal cell is geneticallyengineered with an expression vector comprising:
a suitable promoter;
an internal ribosome entry site (IRES);
a first nucleotidic sequence encoding a suitable selectable marker;
a second nucleotidic sequence encoding for the protein of interest; and
a retroviral long terminal repeat (LTR) sequence flanking at 5′ and/or 3′ ends of the vector;
   wherein the first and second nucleotidic sequences are operably linked one to the other separated by the IRES, and the selectable marker indicating transgenic cells capable of expressing the second nucleotidic sequence.

The patient may. be an immunocompetent patient.

Another object of the present invention is to provide a method of preparing a transgenic bone marrow stromal cell for delivery of a protein of interest into a patient comprising the steps of:
a) providing an isolated stromal cell and culturing the cell in vitro;
b) introducing an expression vector into the isolated marrow stromal cell, wherein the expression vector comprises:
   a suitable promoter;
   an internal ribosome entry site (IRES);
   a first nucleotidic sequence encoding a suitable selectable marker;
   a second nucleotidic sequence encoding for the protein of interest; and
   a retroviral long terminal repeat (LTR) sequence flanking at 5′ and/or 3′ ends of the vector;
wherein the first and second nucleotidic sequences are operably linked to and separated by the IRES, and the selectable marker indicating transgenic cells capable of expressing the second nucleotidic sequence.

Another object of the present invention is to provide a method of introducing and expressing a foreign nucleotidic sequence into a patient comprising the step of:
a) providing an isolated bone marrow stromal cell and culturing the cell in vitro;
b) introducing an expression vector into the isolated stromal cell, wherein the expression vector comprises:
   a suitable promoter;
   an internal ribosome entry site (IRES);
   a first nucleotidic sequence encoding a suitable selectable marker;
   a second nucleotidic sequence encoding for the protein of interest; and
   a retroviral long terminal repeat (LTR) sequence flanking at 5′ and/or 3′ ends of the vector;
wherein the first and second nucleotidic sequences are operably linked to and separated by the IRES, and the selectable marker indicating transgenic cells capable of expressing the second nucleotidic sequence; and
c) implanting the trangenic stromal cell of step b) into an a patient, wherein the implanted cells produce and secrete the protein of interest.

Another object of the present invention is to provide an implant containing cells for modulating tissue synthesis, tissue repair and/or endogenous product synthesis in a patient, the implant comprising a matrix containing viable bone marrow stromal cells as defined in claim 1, dispersed therein.

The modulation may be revitalization, stimulation, induction, or inhibition of tissues synthesis, tissue repair and/or endogenous product synthesis.

In accordance with the present invention there is provided an implant, wherein the transgenic cells are genetically transformed with an expression vector comprising:
a suitable promoter;
an internal ribosome entry site (IRES);
a first nucleotidic sequence encoding a suitable selectable marker; and/or
a nucleotidic sequence of interest encoding for the protein of interest; and
a retroviral long terminal repeat (LTR) sequence flanking at 5′ and/or 3′ ends of the vector;
wherein the first and nucleotidic sequences of interest are operably linked one to the other separated by the IRES, and the selectable marker indicating transgenic cells capable of expressing the nucleotidic sequence of interest.

Another object of the present invention is to provide a method of modulating tissue synthesis, tissue repair and/or endogenous product synthesis in a patient comprising the steps of:
a) providing an isolated bone marrow stromal cell and culturing the cell in vitro;
b) colonizing a biocompatible matrix with the stromal cells of step a) ; and
   implanting the colonized matrix of step b) into a patient, wherein the implanted colonized matrix allows for colonizing stromal cells to modulate tissue synthesis, tissue repair and/or endogenous product synthesis in the patient.

In accordance with the present invention there is provided a matrix that may be selected from the group consisting of chitosan, glycosaminoglycan, chitin, ubiquitin, elastin, polyethylen glycol, polyethylen oxide, vimentin, fibronectin, collagen, derivatives thereof, and combination thereof.

The modulation may be revitalization, stimulation, induction, or inhibition of tissues synthesis, tissue repair and/or endogenous product synthesis.

Another object of the present invention is to provide a method by which hypoxic stimulation of MSCs in vitro enhandes their angiogenic properties in vivo.

Another object of the present invention is to provide with a method allowing tissue synthesis defined as angiogenesis or arteriogenesis.

The product may be selected from the group consisting of lipids, peptides, hormones, glucides, and cytokines.

Stromal cells of the present invention may further be genetically engineered, which may be transgenic cells.

Another object of the present invention is to provide transgenic cells genetically transformed with an expression vector comprising:
a suitable promoter;
an internal ribosome entry site (IRES);
a first nucleotidic sequence encoding a suitable selectable marker; and/or
a nucleotidic sequence of interest encoding for the protein of interest; and
   a retroviral long terminal repeat (LTR) sequence flanking at 5′ and/or 3′ ends of the vector;
wherein the first and nucleotidic sequences of interest are operably linked one to the other separated by the IRES, and the selectable marker indicating transgenic cells capable of expressing the nucleotidic sequence of interest.

The patient of the present invention may be a human or an animal.

The expression of the present invention may be a bicistronic retroviral vector or a vector made with DNA or RNA.

The selectable marker may be selected from the group consisting of drug resistance, enhanced green fluorescent protein (EGFP), and β-galactosidase.

The protein of interest may be autologous or heterologous, and may be selected from the group consisting of cytokine, interleukin, growth hormones, hormones, blood factors, marker proteins, immunoglobulins, antigens, releasing hormone, anticancer product, antitumor product, antiviral product, antiretroviral product, an antisense, an antiangiogenic product, an angiogenic product, a replication inhibitor, erythropoietin, an analog or a fragment thereof.

The promoter may comprise a retroviral or synthetic promoter.

For the purpose of the present invention the following terms are defined below.

The term “genetically-engineered stromal cell” or “transgenic stromal cells” as used herein is intended to mean a stromal cell into which an exogenous gene has been introduced by retroviral infection or other means well known to those of ordinary skill in the art. The term “genetically-engineered” may also be intended to mean transfected, transformed, transgenic, infected, or transduced.

The term “ex vivo gene therapy ” is intended to mean the in vitro transfection or retroviral infection of stromal cells to form transfected stromal cells prior to implantation into a mammal.

As used herein, “exogenous genetic material” refers to a nucleic acid or an oligonucleotide, either natural or synthetic, that is not naturally found in bone marrow stromal cells; or if it is naturally found in the cells, it is not transcribed or expressed at biologically significant levels by bone marrow stromal cells. Thus, “exogenous genetic material” includes, for example, a non-naturally occurring nucleic acid that can be transcribed into anti-sense RNA, as well as a “heterologous gene” (i.e., a gene encoding a protein which is not expressed or is expressed at biologically insignificant levels in a naturally-occurring bone marrow stromal cell). To illustrate, a synthetic or natural gene encoding human erythropoietin (EPO) would be considered “exogenous genetic material” with respect to human bone marrow stromal cells since the latter cells do not naturally express EPO; similarly, a human interleukin-2 gene inserted into a bone marrow stromal cell would also be an exogenous gene to that cell since peritoneal bone marrow stromal cells do not naturally express interleukin-2 at biologically significant levels. Still another example of “exogenous genetic material” is the introduction of only part of a gene to create a recombinant gene, such as combining an inducible promoter with an endogenous coding sequence via homologous recombination.

As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material which has a beneficial effect on the mammalian recipient. Thus, “therapeutic agent” embraces both therapeutic and prophylactic molecules having nucleic acid (e.g., antisense RNA) and/or protein components.

As used herein, “acquired pathology” refers to a disease or syndrome manifested by an abnormal physiological, biochemical, cellular, structural or molecular biological state.

The term “therapeutic agent” as used herein may include, but is not limited to proteins under native form, as well as their functional equivalents.

As used herein, the term “functional equivalent peptide or protein” refers to a molecule (e.g., a peptide or protein), that has the same or an improved beneficial effect of a mammalian recipient, acting as a therapeutic agent of which is it deemed a function equivalent to endogenous peptides or proteins. It will be appreciated by one of ordinary skill in the art, functionally equivalent proteins can be produced by recombinant techniques, e.g., by expressing a “functionally equivalent DNA”.

As used herein, the term “functionally equivalent DNA” refers to a non-naturally occurring DNA that encodes a therapeutic agent. However, due to the degeneracy of the genetic code, more than one nucleic acid can encode the same therapeutic agent. Accordingly, the instant invention embraces therapeutic agents encoded by naturally occurring DNAs, as well as by non-naturally-occurring DNAs that encode the same protein as encoded by the naturally occurring DNA.

The above-disclosed therapeutic agents and conditions amenable to gene replacement therapy are merely illustrative and are not intended to limit the scope of the instant invention. The selection of a suitable therapeutic agent for treating a known condition is deemed to be within the scope of one of ordinary skill of the art without undue experimentation.

The exogenous genetic material (e.g., a cDNA encoding one or more therapeutic proteins) is introduced into the bone marrow stromal cell ex vivo or in vivo by genetic transfer methods, such as transfection or transduction, to provide a genetically modified bone marrow stromal cell. Various expression vectors (i.e., vehicles for facilitating delivery of exogenous genetic material into a target cell) are known to one of ordinary skill in the art.

In contrast, “transduction of bone marrow stromal cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced bone marrow stromal cell. A bone marrow stromal cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence which works contiguous with the coding sequence (in cis) to change the basal transcription level dictated by the promoter. Preferably, the exogenous genetic material is introduced Into the bone marrow stromal cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

The term ” stromal cells ” as used herein is intended to mean marrow-derived fibroblast-like cells defined by their ability to adhere and proliferate in tissue-culture treated petri dishes with or without other cells and/or elements found in loose connective tissue, including but not limited to, endothelial cells, pericytes, macrophages, monocytes, plasma cells, mast cells, adipocytes, etc.

The term “tissue-specific” as used herein is intended to mean the cells that form the essential and distinctive tissue of an organ as distinguished from its supportive framework.

The term “implant” as used herein is intended to mean a three dimensional matrix composed of any material and/or shape that (a) allows cells to attach to it (or can be modified to allow cells to attach to it); and (b) allows cells to grow in more than one layer and proliferates to be dispersed therein. This support is inoculated with stromal cells to form the implant stromal matrix. A stromal implant which has been inoculated with tissue-specific cells and cultured. In general, the tissue specific cells used to inoculate the implant stromal matrix may include the “stem” cells (or “reserve” cells) for that tissue; i.e., those cells which generate new cells that will mature into the specialized cells that form the parenchyma or other structures of a targeted tissue. The term “implant” may also mean introduction of the bioactive material/matrix by means of injection, surgery, catheters or any other means whereby cells producing bioactive material or participate to regeneration to tissues or endogenous product synthesis.

The term “implant stromal matrix” as used herein is intended to mean a three dimensional matrix which has been inoculated with stromal cells. Whether confluent or subconfluent, stromal cells according to the invention continue to grow and divide. The stromal matrix will support the growth of tissue-specific cells later inoculated to form the three dimensional cell culture.

The term “revitalize” as used herein is intended to mean restore vascularization to tissue having been injured. The repair of tissues may be done by neo-synthesis. The term “injury” as used herein means a wound caused by ischemia, infarction, surgery, irradiation, laceration, toxic chemicals, viral infection or bacterial infection.

The term “controlled release implant” means any composition that will allow the slow release or in situ synthesis of a bioactive substance that is mixed or admixed therein. The matrix containing cells can be a solid composition, a porous material, or a semi-solid, gel or liquid suspension containing the bioactive substance.

The term “bioactive material” means any angiogenic composition that will promote vascularization and revitalization of tissue when used in accordance with the present invention.

The term “cytokine” as used herein may include but is not limited to growth factors, interleukins, interferons and colony stimulating factors. These factors are present in normal tissue at different stages of tissue development, marked by cell division, morphogenesis and differentiation. Among these factors are stimulatory molecules that provide the signals needed for in vivo tissue repair. These cytokines can stimulate conversion of an implant into a functional substitute for the tissue being replaced. This conversion can occur by mobilizing tissue cells from similar contiguous tissues, e.g., from the circulation and from stem cell reservoirs. Cells can attach to the prostheses which are bioabsorbable and can remodel them into replacement tissues.

### BRIEF DESCRIPTION OF THE DRAWINGS

FIG. 1 shows a schematic illustration of the retroviral plasmid construct pEpo-IRES-EGFP;

FIG. 2 illustrates the erythropoietin (Epo) secretion by gene-modified mouse marrow stroma prior to implantation;

FIG. 3 illustrates the hematocrit of mice implanted with Epo-secreting marrow stroma;

FIG. 4 illustrates a dose-response between the number of Epo-secreting marrow stromal cells implanted in mice and the increase in hematocrit;

FIG. 5 shows a southern blot analysis of Epo-IRES-EGFP Transduced Mouse Marrow Stroma;

FIG. 6 illustrates a dose-response between the number of implanted Epo-secreting MSCs and the hematocrit increase;

FIG. 7 illustrates the plasma Epo concentration of mice implanted with genetically. engineered MSCs;

FIG. 8 illustrates a section of muscles showing the implantation of stromal cells;

FIG. 9 illustrates the hematocrit level (HCT) through 4 weeks after implantation of engineered stromal cells in mice;

FIG. 10 illustrates the anglogenic response in murine Matrigel™ assay induced by bFGF, murine VEGF 165 and MSCs at 28 days post implantation;

FIGS. 11 illustrate the angiogenic response in murine Matrigel™ assay induced by bFGF, murine VEGF 165 and MSCs at 14 days post implantation;

FIG. 12 illustrates the level of plasma. Epo after Implantation of Matrigel™ containing different quantities of Epo secreting engineered MSCs;

FIG. 13, Illustrates Hematocrit (Hct) and plasma Epo concentration of mice following Intraperitoneal implantation with mEpo-secreting marrow stromal cells;

FIG. 14 illustrates Hematocrit (Hct) and plasma Epo concentration of mice following subcutaneous Implantation with mEpo-secreting marrow stromal cells embedded in Matrigel ™;

FIG. 15 illustrates in vivo differentiation of Matrigel-embedded Epo-secreting marrow stromal cells into CD31+ endothelial cells;

FIG. 16 illustrates long-term hematocrit of mice following subcutaneous implantation of mEpo-secreting marrow stromal cells with or without Matrigel; and

FIG. 17 illustrates long-term hematocrit of mice following subcutaneous implantation of mEpo-secreting marrow stromal cells embedded in a human biocompatible type I bovine collagen matrix.

### DETAILED DESCRIPTION OF THE INVENTION

In accordance with the present invention, there is provided an autologous cellular vehicle for transgene delivery which is (i) abundant and available in humans of all age groups, (ii) harvested with minimal morbidity and discomfort, (iii) manipulated and genetically engineered with relative efficiency and lastly, (iv) easy to reimplant in the donor. Bone marrow stromal cells fulfill these criteria.

In another embodiment of the invention, there is provided a recombinant protein delivery system and method of preparation thereof. When whole marrow aspirates are placed in culture, two populations distinguish themselves promptly: (i) “adherent” fibroblast-like cells and (ii) a mixture of free-floating” hematopoietic cells. The fibroblast-like cells will give rise to colonies also known as Colony Forming Units-Fibroblast (CFU-F). CFU-Fs—hereafter referred to as marrow stromal cells (MSCs), can be implanted directly in organs—such as brain—without need of “conditioning” regimens. Widespread, multiorgan engraftment occurs following intravenous or intraperitoneal infusion of stromal cells in mice that may optionally receive low-dose irradiation. Furthermore, large number of stromal cells can be re-infused intravenously without adverse effect in humans, and clinical protocols examining engraftment of allogeneic as well as genetically-marked autologous stromal cells are underway.

In one embodiment of the genetically engineered stromal cells of the present invention, a gene encoding for valuable therapeutic protein is introduced. Among these proteins, there is the erythropoietin. Erythropoietin (Epo), a glycoprotein hormone, is the main regulator of erythropoiesis in mammalian blood. Recombinant human Epo is commonly used for the treatment of Epo-responsive anemias that may arise as a consequence of hemoglobinopathies, chronic renal failure, cancer, or AIDS. However, recombinant protein administration is often limited by the suboptimal pharmacokinetics, the need for repeated incommodious injections and hence poor patient compliance, as well as the cost to the patient. The genetically-engineered bone marrow stromal cells and gene therapy approach of the present invention allows to overcome these obstacles and obviate the requirement for recombinant protein administration by imparting systemic secretion of Epo. Marrow stromal cells are useful as vehicles for beneficial gene products as they can easily be isolated from bone marrow aspirates, expanded in vitro, transduced with viral vectors, and maintained in vivo.

Autologous marrow stromal cells(MSCs) are expanded and/or treated to enter active cell cycling in vitro by methods well-known to those skilled in the art.

The invention also features a method of ex vivo gene therapy in which the BSCs are induced to proliferate for retroviral vector integration and then induced to become quiescent prior to introduction into a mammal.

In another embodiment of the present invention, a method is used for treating an inherited, an acquired, or a metabolic deficiency-in a mammal (such as a human). For example, the transfected MSCs may contain expressible DNA for the production of antisense RNA in order to reduce the expression of an endogenous gene of the mammal.

In another embodiment of the present invention, the transfected MSCs may contain DNA encoding a protein capable of preventing or treating an inherited or acquired disease (e.g., Factor VIII deficiency in hemophilia, cystic fibrosis, and adenosine deaminase deficiency). Infused cells or their progeny preferably contain a marker such that the infused cells are observable in a population of host cells for the purpose of selecting most desirable cell lines before transplantation into a host human or animal, or even to measuring the level of engraftment. The gene of interest that is incorporated in the vectors of the invention may be any gene, which produces an hormone, an enzyme, a receptor or a drug(s) of interest.

Another embodiment of the present invention is to provide a class of bone marrow stromal cells genetically-engineered with bicistronic retroviral vectors. The retroviral vectors provided for contain (1) 5′ and 3′ LTRs derived from a retrovirus of interest, as the Vesicular Stomatitis Virus, of the Moloney murine leukemia virus; (2) an insertion site for a gene of interest; (3) a selectable gene marker, as the gene encoding for the cytidine deaminase, β-galactosidase or any other useful marker, and (4) an internal ribosome entry site (IRES) between the marker gene and the gene of interest. The retrovirus vectors of the subject invention may not contain a complete gag, env, or pol gene, so that the retroviral vectors are incapable of independent replication in target cells.

In one embodiment of the present invention, there is provided a method of genetically engineering mammalian cells that has proven to be particularly useful is by means of retroviral vectors. Retroviral vectors are produced by genetically manipulating retroviruses.

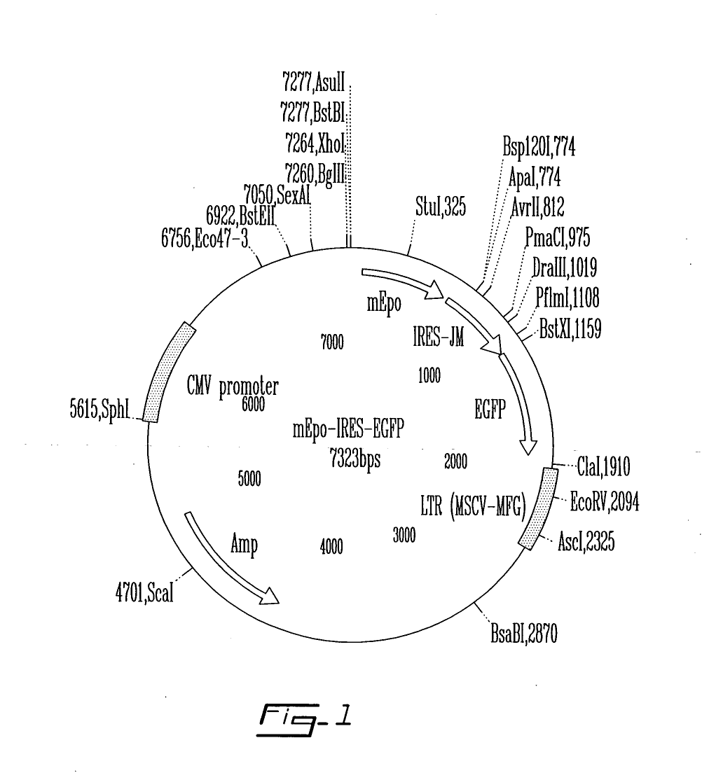

In still another embodiment, retroviruses of the present invention are RNA viruses; that is, the viral genome is RNA. This genomic RNA is, however, reverse transcribed into a DNA copy which is integrated stably and into the chromosomal DNA of transduced cells. This stably integrated DNA copy is referred to as a provirus and is inherited by daughter cells as any other gene. As shown in FIG. 1, the wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAS.

Retroviral vectors are particularly useful for modifying mammalian cells because of the efficiency with which the retroviral vectors “infect” target cells and integrate into the target cell genome. Additionally, retroviral vectors are useful because the vectors may be based on retroviruses that are capable of infecting mammalian cells from a wide variety of species and tissues.

The ability of retroviral vectors to insert into the genome of mammalian cells have made them particularly promising candidates for use in the genetic therapy of genetic diseases in humans and animals. Genetic therapy typically involves (1) adding new genetic material to patient cell in vivo, or (2) removing patient cells from the body, adding new genetic material to the cells and reintroducing them into the body, i.e., in vitro gene therapy.

In another embodiment of the present invention, the mammalian recipient has a condition that is amenable to gene replacement therapy.

The condition amenable to gene replacement therapy alternatively can be a genetic disorder or an acquired pathology that is manifested by abnormal cell proliferation, e.g., cancers arising in or metastasizing to the coelomic cavities. According to this embodiment, the instant invention is useful for delivering a therapeutic agent having anti-neoplastic activity (i.e., the ability to prevent or inhibit the development, maturation or spread of abnormally growing cells), to tumors arising in or metastasizing to the coelomic cavities, (e.g., ovarian carcinoma, mesothelioma, colon carcinoma).

Alternatively, the condition amenable to gene replacement therapy is a prophylactic process, i.e., a process for preventing disease or an undesired medical condition. Thus, the instant invention embraces a bone marrow stromal cell expression system for delivering a therapeutic agent that has a prophylactic function (i.e., a prophylactic agent) to the mammalian recipient. Such therapeutic agents (with the disease or undesired medical condition they prevent appearing in parentheses) include: growth hormone (aging); thyroxine (hypothyroidsm); and agents which stimulate, e.g., gamma-interferon, or supplement, e.g., antibodies, the immune system response (diseases associated with deficiencies of the immune system).

In another embodiment of the present invention, a naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88: 4626-4630 (1991)), adenosine deaminase, phosphoglycerol kinase (PGK), pyruvate kinase, phosphoglycerol mutase, the .beta.-actin promoter, and other constitutive promoters known to those of skill in the art. In addition, many viral promoters function constitutively in eucaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter Is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak) it is possible to control both the existence and level of expression of a therapeutic agent in the genetically modified bone marrow stromal cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified bone marrow stromal cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the bone marrow stromal cell; (3) the number of transduced/transfected, bone marrow stromal cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified bone marrow stromal cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector preferably includes a selection gene, for example, a neomycin resistance gene, for facilitating selection of bone marrow stromal cells that have been transfected or transduced with the expression vector.

Alternatively, the bone marrow stromal cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

In still another embodiment of the invention, the therapeutic agent can be targeted for delivery to an extracellular, intracellular or membrane location. If it is desirable for the gene product to be secreted from the bone marrow stromal cells (e.g., to deliver the therapeutic agent to the lymphatic and vascular systems), the expression vector is designed to include an appropriate secretion “signal” sequence for secreting the therapeutic gene product from the cell to the extracellular milieu. If it is desirable for the gene product to be retained within the bone marrow stromal cell, this secretion signal sequence is omitted. In a similar manner, the expression vector can be constructed to include “retention” signal sequences for anchoring the therapeutic agent within the bone marrow stromal cell plasma membrane. For example, all membrane proteins have hydrophobic transmembrane regions that stop translocation of the protein in the membrane and do not allow the protein to be secreted. The construction of an expression vector including signal sequences for targeting a gene product to a particular location is deemed to be within the scope of one of ordinary skill in the art without the need for undue experimentation.

The selection and optimization of a particular expression vector for expressing a specific gene product in an isolated bone marrow stromal cell is accomplished by obtaining the gene, preferably with one or more appropriate control regions (e.g., promoter, insertion sequence); preparing a vector construct comprising the vector into which is inserted the gene; transfecting or transducing cultured bone marrow stromal cells in vitro with the vector construct; and determining whether the gene product is present in the cultured cells.

In accordance with the present invention, there is provided implants containing cultured bone marrow stromal cells can directly promote and participate in neo-angiogenesis in vivo.

In one embodiment of the invention, there is provided an implant allowing vascular differentiation, and likely therapeutic benefit, of stromal cells which is dependent upon embedding in a matrix that may contain laminin, collagen IV, entactin, heparan sulfate proteoglycan, matrix metalloproteinases, growth factors, and other components of interest.

In another embodiment, there is a retroviral and expression vector engineering of bone marrow stromal cells to secrete products that are capable of participating to tissue regeneration, tissue synthesis and tissue repair.

In another embodiment, the implant of the invention includes implantation into a patient of a matrix containing cells that participate to the neo-synthesis of surrounding tissues, as for example but without limitation, to angiogenesis.

Genetic engineering of MSCs with other therapeutic transgenes and/or anti-sense vectors may alter the phenotype of the cells in a manner leading to enhanced angiogenic effect in vivo.

In one embodiment of the invention, genetic engineering of cells with non-viral vectors for similar effect may also be feasible.

In another embodiment of the present invention, there is provided bone marrow stromal cells and their geneticallyengineered counterparts that promote neovascularization. in ischemic organs. Cultured autologous stromal cells embedded in matrix can be used to grow new functional blood vessels for treatment of vascular insufficiency.

There is also provided with the present invention marrow stromal cells and their erythropoietin (Epo)-secreting counterparts are of therapeutic utility in vascular insufficiency, including myocardial, peripheral limb and cerebral ischemia. A role for marrow stromal cells in angiogenesis by differentiating into endothelial cells and/or other cellular types.

Another embodiment of the invention is to provide a method for cell therapy of vascular insufficiency and for induction of angiogenesis by implanting genetically modified autologous cells to secrete an angiogenic factor.

Also is provided with the invention the use of erythropoietin to induce angiogenesis in ischemic organs through production by transgenic stromal cells implanted into a matrix.

In another embodiment of the present invention, there is provided a biodegradable implant which has significantly enhanced biocompatibility in that (1) blood compatibility is substantially improved, (2) immunogenicity is minimized, and (3) the matrix is enzymatically degraded to endogenous, nontoxic compounds. The process for making the novel implant represents a further advance over the art in that, during synthesis, one can carefully control factors such as hydrophilicity, charge and degree of cross-linking. By varying the composition of the matrix as it is made, one can control the degradation kinetics of the hydrogel formulation and the overall timed-release profile.

In one embodiment of the present invention, there is provided an implant for “Therapeutic Neo-angiogenesis”. Different approaches have been explored, among them: administration of anglogenic factors or stimulation of their endogenous secretion (e.g. by drugs, trauma, inflammation, or mast cells stimulation); angiogenic factor-coding gene transfer and cell transplantation. Angiogenesis refers to the formation of new blood vessels from pre-existing ones by sprouting from small venules. Embryologically, endothelial cells originate by differentiation from mesodermal hemangioblasts. Endothelial cell progenitors (EC), also known as angioblasts, can be found circulating in human blood. These cells can differentiate into endothelial cells and can participate in the process of angiogenesis. In animal models of ischemia, heterologous, homologous, and autologous EC progenitors incorporated into sites of active angiogenesis.

The origin of these cells was shown to be the bone marrow. It is considered that cells with angiogenic properties may be harnessed for therapeutic use for rebuilding or adding new blood vessels to ischemic anatomic compartments such as the heart, brain and peripheral limbs.

The use of cells for cell therapy applications alleviate the need for fetal or allogeneic donors and the attendant requirement for pharmacological immunosuppression. The issue then arises of the nature and source of autologous cells to be used for neo-angiogenic purposes. A desirable cellular vehicle for neo-angiogenic cell therapy may be (i) abundant and available in humans of all age groups, (ii) harvested with minimal morbidity and discomfort, (iii) cultured with reasonable efficiency and lastly, (iv) easy to reimplant in the donor. Bone marrow stromal cells of the present Invention fulfill these criteria. Furthermore, we have preliminary data that strongly supports the fact that marrow stromal cells are capable of contributing to formation of functional vascular structures in vivo.

When whole marrow aspirates are placed in culture, two populations distinguish themselves promptly: (i) “adherent” fibroblast-like cells and (ii) a mixture of free-floating” hematopoietic cells. The fibroblast-like cells will give rise to colonies also known as Colony Forming Units-Fibroblast (CFU-F). CFU-Fs—are considered here to as marrow stromal cells (MSCs).

In vitro and in vivo studies showed that MSCs are pleuripotent and have the ability to differentiate into osteoblasts, chondroblasts, fibroblasts, adipocytes, skeletal myoblasts and cardiomyocytes. The present invention shows that cultured MSCs when injected into the myocardium may undergo milieu-dependent differentiation into cardiomyocytes. It is also shown that implantation of autologous bone marrow cells in rat ischemic heart model will enhance angiogenesis presumably arising from the secretion of interleukin-1β (IL-1β) and Cytokine-Induced Neutrophil Chemoattractant (CINC) from marrow stromal cells.

Genetic reprogramming of cultured cell lines with recombinant DNA is routinely carried out as a mean to decipher the molecular mechanisms of disease. Gene transfer and expression is an extremely powerful tool which may be exploited for therapeutic purposes. Strategies can be devised where the introduction of synthetic genetic information will alter the phenotype of cultured cells.

In still another embodiment of the invention, there is provided a gene therapy method for the treatment of disease that utilizes synthetic genetic material as a pharmacological agent. The common denominator to all cell and gene therapy strategies is to “reprogram” the behavior of cells for therapeutic effect.

An important issue to be addressed for “transgenic cell therapy” is the development of a practical cellular vehicle for secretion of angiogenic factors in humans with vascular insufficiency. Autologous MSCs may be desirable because they can be genetically engineered.

It is an embodiment that MSCs genetically-engineered to express bacterial beta-galactosidase can be implanted directly in organs—such as brain, muscle and heart—without need of “conditioning” regimens.

In one embodiment of the invention, genetically engineered stromal cells may serve as a cellular vehicle for therapeutic proteins in vivo. It is a property of the invention that MSCs engineered may secrete an angiogenic factor and enhance the local neovascularization associated with their use. There are several angiogenic factors currently under investigation for therapeutic angiogenesis, including VEGF, bFGF, α-TGF, β-TGF, and Hepatocyte growth factor and many have been extensively explored as part of gene therapy strategies for treatment of ischemic disease. Erythropoietin has recently been found to have angiogenic effects and its therapeutic neo-angiogenic properties remain unexplored.

In another embodiment of the present invention, there is provided an implant that allows for delivery of erythropoietin. Erythropoiefin (EPO) is a glycoprotein hormone produced by the kidney and is the major humoral regulator of red blood cell production. The main haematopoietic effects of EPO are the stimulation of early erythroid cells proliferation and the differentiation of late precursors. EPO also prevents rapid apoptosis of erythroid cells and has a proven regulatory effect on megakaryocytes and their progenitors. The relationship between EPO and angiogenesis was initially suspected on the basis of the common developmental origin of both haematopoietic cells and endothelial cells from the hemangioblast. Both cell types were found to share common surface antigens e.g. CD31, CD34, and MB1. Endothelial cells can express the EPO receptor and it has been shown that recombinant human EPO (rhEPO) has a mitogenic and positive chemotactic effect on endothelial cells. rhEPO will stimulate angiogenesis in vitro as well as in the chick embryo chorioallantoic membrane (CAM) assay. EPO has also been found to play a physiological angiogenic role in vivo, where estrogen dependent production of EPO in the mouse uterus elicits an angiogenic effect. It is shown that high local concentrations lead to uterus-restricted angiogenesis without concurrent erythrocytosis. In patients chronically receiving recombinant human EPO for anemia (like renal failure patients) angiogenic side effects (e.g. aggravation of diabetic retinopathy or growth of latent neoplasm) have not been reported. This suggests that the EPO-mediated angiogenic effect can be achieved locally with minimal or no systemic neo-angiogenic effect. Furthermore, EPO might have a supplementary protective role against ischemic damage. This was at least proven in the brain, where it was found that in mice treated with recombinant EPO 24 hours before induction of cerebral ischemia had a significant reduction in infarct volume.

Examples of tissues which can be repaired and/or reconstructed using the implants and implant compositions described herein include nervous tissue, skin, vascular tissue, muscle tissue, connective tissue such as bone, cartilage, tendon, and ligament, kidney tissue, and glandular tissue such as liver tissue and pancreatic tissue. In one embodiment, the implants and implant compositions seeded with tissue specific cells are introduced into a recipient, e.g., a mammal, e.g., a human. Alternatively, the seeded cells which have had an opportunity to organize into a tissue in vitro and to secrete tissue specific biosynthetic products such as extracellular matrix proteins and/or growth factors which bond to the implants and implant compositions are removed prior to introduction of the implants and implant compositions into a recipient.

Different biopolymers can be furnished by natural sources. Collagen or combinations of collagen types can be used in the implants and implant compositions described herein. A desired combination of collagen types includes collagen type I, collagen type III, and collagen type IV. Preferred mammalian tissues from which to extract the biopolymer include entire mammalian tissues or fetuses, e.g., porcine fetuses, dermis, tendon, muscle and connective tissue. As a source of collagen, fetal tissues are advantageous because the collagen in the fetal tissues is not as heavily crosslinked as in adult tissues. Thus, when the collagen is extracted using acid extraction, a greater percentage of intact collagen molecules is obtained from fetal tissues in comparison to adult tissues. Fetal tissues also include various molecular factors which are present in normal tissue at different stages of animal development.

In one embodiment of the invention, there is provided production or delivery of cellular matrix proteins. The extracellular matrix includes extracellular matrix proteins. For example, extracellular matrix proteins obtained from skin include transforming growth factor beta-1, platelet-derived growth factor, basic fibroblast growth factor, epidermal growth factor, syndecan-1, decorin, fibronectin, collagens, laminin, tenascin, and dermatan sulfate. Extracellular matrix proteins from lung include syndecan-1, fibronectin, laminin, and tenascin. The extracellular matrix protein can also include cytokines, e.g., growth factors necessary for tissue development.

In another embodiment of the invention, there is provided encapsulated live cells, organelles, or tissue have many potential uses. For example, within a semipermeable implant, the encapsulated living material can be preserved in a permanent sterile environment and can be shielded from direct contact with large, potentially destructive molecular species, yet will allow free passage of lower molecular weight tissue nutrients and metabolic products. Thus, the development of such an encapsulation technique could lead to systems for producing useful hormones such as erythropoietin, or others. In such systems, the mammalian tissue responsible for the production of the material would be encapsulated in a manner to allow free passage of nutrients and metabolic products across the implant, yet prohibit the passage of bacteria. As implant permeability may be controlled, it is possible that this approach could also lead to artificial organs, or precursor organs, which could be implanted in a mammalian body, e.g., a diabetic, without rejection and with controlled hormone release, e.g., insulin release triggered by glucose concentration. Vascular tissues may be regenerated with such method of the present invention.

Growth factors necessary for cell growth are attached to structural elements of the extracellular matrix. The structural elements include proteins, e.g., collagen and elastin, glycoproteins, proteoglycans and glycosaminoglycans. The growth factors, originally produced and secreted by cells, bind to the extracellular matrix and regulate cell behavior in a number of ways. These factors include, but are not limited to, epidermal growth factor, fibroblast growth factor (basic and acidic), insulin-like growth factor, nerve growth-factor, mast cell-stimulating factor, the family of transforming growth factor beta, platelet-derived growth factor, scatter factor, hepatocyte growth factor and Schwann cell growth factor.

The extracellular matrix may play also an instructive role, guiding the activity of cells which are surrounded by it or which are dispersed into it. Since the execution of cell programs for cell division, morphogenesis, differentiation, tissue building and regeneration depend upon signals emanating from the extracellular matrix, three-dimensional scaffolds, such as collagen implants, may be enriched with actual matrix constituents or secreted by stromal cells, which may exhibit the molecular diversity and the microarchitecture of a generic extracellular matrix, and of extracellular matrices from specific tissues.

The present invention will be more readily understood by referring to the following examples, which are given to illustrate the invention rather than to limit its scope.

### EXAMPLE I

### Erythropoietin Secretion by Rat Bone Marrow Stromal Cells Following Retroviral Gene Transfer

Erythropoiesis in mammalian bone marrow is primarily regulated by the glycoprotein hormone, erythropoietin (Epo). Recombinant human Epo is commonly utilized for the treatment of Epo-responsive anemias. The administration of recombinant proteins, such as Epo, in acquired and inherited disorders, is often characterized by their suboptimal pharmacokinetics, the requirement for repeated incommodious injections, and cost to the patient. These impediments have incited the development of novel cell and gene therapy strategies. One approach is to use gene-modified bone marrow stromal cells, also referred to as mesenchymal stem cells (MSCs), to impart sustained systemic secretion of a therapeutic protein. MSCs are appealing as vehicles for beneficial gene products as they can easily be isolated from bone marrow aspirates, expanded in vitro, transduced with viral vectors, and maintained in vivo. One object of the present study was to investigate if primary rat MSCs can be engineered to express and secrete murine Epo in vitro by means of retroviral gene transfer. Retroviral vectors as gene delivery systems provide the advantage of stable transgene expression through their ability to integrate into the cellular genome, thereby ensuring that gene-modified cells and their progeny will secrete the therapeutic protein. A bicistronic vesicular stomatitis virus G pseudotyped retroviral vector containing the mouse Epo cDNA and the green fluorescent protein (GFP) reporter gene was generated and utilized to transfect 293GPG packaging cells. The ensuing mixed population of retrovirus-producing cells was 76% GFP positive, as determined by flow cytometry analysis. Filtered viral supernatant served to transduce primary rat MSCs at a multiplicity of infection (MOI) of 12 infectious particles per cell, yielding 11.6±1.5 (mean±S.D.; n=3) % GFP positive MSCs. Significant levels of Epo in the media of these transduced cells were detected by enzyme-linked immunosorbent assay (ELISA). Gene-modified MSCs secreted 5.2±0.8 (mean±S.D.; n=3) units of Epo per 10⁶ cells in 24 hrs, versus a background of <0.3 in untransduced MSCs (P<0.005). Moreover, the retroviral transduction of A549 human lung carcinoma cells has been performed and noted a strong dose-effect relationship (r>0.97) between the MOI and the degree of Epo secretion. In conclusion, the present data indicate that MSCs, consequent to retrovirus-mediated gene transfer, can effectively release Epo in vitro. Future studies will comprise the implantation of the genetically altered MSCs in anemic rodents and the exploration of an inducible expression system to control the level of expression and secretion of Epo. The potential of MSCs as vehicles for the in vivo secretion of therapeutic proteins extends to all diseases where clinical improvement is feasible via the delivery of a specific gene product.

### EXAMPLE II

### High-Level Erythropoietin Production from Genetically Engineered Bone Marrow Stroma Implanted in Non-Myeloablated, Immunocompetent Mice

Autologous bone marrow stromal cells are appealing as a cellular vehicle for delivery of therapeutic proteins. They can be readily harvested from donors without the need of mobilization regimens, are easily expanded in tissue culture and are amenable to genetic engineering with integrating viral vectors. Their penultimate use in transgenic adoptive cell therapy of disease will be dependent upon their capability to engraft in non-myeloablated, immunocompetent recipients. To test this, it was determined whether intra-peritoneal implantation of isogenic stromal cells retrovirally-engineered to secrete mouse erythropoietin (mEpo) would lead to a rise of the number of red blood cells with time. The mouse Epo cDNA into a bicistronic retroviral vector comprising the green fluorescent protein (GFP) reporter gene downstream of an internal ribosome entry site (IRES) was cloned. The resulting construct was stably transfected into GP+E86 packaging cells, consequently generating Epo-GP+E86 cells producing ˜2.5×10⁵ infectious particles per ml, as determined by titer assay on NIH 3T3 cells. Primary bone marrow stromal cells from C57BI/6 mice were transduced with retroparticles from Epo-GP+E86 cells once a day for 3 consecutive days and subsequently allowed to expand in culture for ˜2 months. These genetically engineered cells were revealed to secrete ˜200 mU of Epo per 10⁶ cells per 24 hours, as determined by enzyme-linked immunosorbent assay (ELISA). In addition, 54% of this Epo-transduced stromal cell population expressed GFP, as ascertained by flow cytometry analysis. Provirus integration and lack of rearrangement in transduced cells was confirmed by Southern Blot analysis of restriction enzyme digested genomic DNA. Three C57BI/6 mice had 107 Epo-secreting marrow stromal cells implanted into their abdominal cavity by intraperitoneal (i.p.) injection. The hematocrit of these recipients rose from a basal level of 53±2% (mean±SEM) to 76±1% within two weeks following implantation and persisted to escalate further attaining a value of 88±1% at 12 weeks post-implantation. A parallel cohort of animals (n=5) received 10⁷ stromal cells engineered with a control retrovector. Their hematocrit remained at basal levels (51 to 57%) throughout the study. In conclusion, these findings strongly support the use of autologous bone marrow stroma as a delivery vehicle for sustained systemic production of recombinant therapeutic proteins in normal immunocompetent animals.

### EXAMPLE III

### Dexamethasone Regulated Erythropoietin Secretion by Bone Marrow Stromal Cells Following Retroviral Gene Transfer

Marrow stromal cells are attractive as a cellular vehicle for the delivery of recombinant proteins, such as erythropoietin (Epo), as they can easily be isolated from bone marrow aspirates, expanded in vitro, transduced with viral vectors, and maintained in vivo. Regulatable expression is vital in therapeutic applications where continuous transgene expression would be deleterious. Marrow stroma can be engineered with a glucocorticoid-inducible retroviral vector developed in our laboratory and that transgene expression is inducible with dexamethasone and repetitively reversible. The objective of the present investigation was to explore this drug-inducible genetic switch to provide “on-demand” secretion of Epo. A retroviral construct has been generated, GRE5mEpoGFP, comprising the mouse Epo cDNA, an internal ribosome entry site, and the green fluorescent protein (GFP) gene, all under the control of an inducible promoter containing 5 glucocorticoid response elements (GRE5) driving transgene expression in transduced cells. This recombinant plasmid DNA was stably transfected into GP+E86 packaging cells and virus-producers were generated. Bone marrow was harvested from the hind leg femurs and tibias of C57BI/6 mice and 5 days later stromal cells were exposed twice per day for 3 consecutive days for each of 2 weeks to retroparticles. At over 72 hrs post-transduction, cells were exposed to 250 nM dexamethasone for 6 successive days. Throughout this interval, media was collected daily from engineered stroma and evaluated by enzyme linked immunosorbent assay (ELISA) for the amount of secreted Epo. GRE5-mEpo-GFP transduced stromal cells were noted to secrete increasing levels of Epo attaining 338±69 mU per 10⁶ cells per 24 hrs (mean±SEM, n=3) following 6 day drug exposure. In the absence of dexamethasone only very low level transcriptional activity, hence little “leakiness”, was observed, precisely 20±2 mU Epo/10⁶ cells/24 hrs. A parallel group of stromal cells was engineered with a control retrovector and likewise exposed to dexamethasone. Epo secretion by these cells remained at normal basal levels, 7±5 mU/10⁶ cells/24 hrs (n=3) throughout the 6 days. These data clearly establish that GRE5-mEpo modified stroma may serve as a cellular vehicle for dexamethasone regulated production of therapeutic levels of erythropoietin in vivo.

### EXAMPLE IV

### Sustained Erythrocytosis Following Intraperitoneal Implantation of Erythropoietin Gene-Modified Autologous Marrow Stroma in Non-Myeloablated, Immunocompetent Mice

Systemic transgene delivery can be accomplished by implanting gene-modified autologous cells via intravenous, intramuscular, intraperitoneal, and subcutaneous administration. Cell types explored as gene delivery vehicles encompass skin fibroblasts, myoblasts, vascular smooth muscle cells, hematopoietic stem cells, lymphocytes, and human umbilical vein endothelial cells However, there are drawbacks associated with the use of these cells in an autologous setting. It is known that skin fibroblasts inactivate introduced vector sequences following transplantation and depending on the age of the donor have limited in vitro proliferation capacities, thus requiring the harvest of considerable quantities of primary cells. Skeletal myoblasts are present in very low amounts in the majority of adult mammals, and their successful growth and transplantation is technically challenging. Vascular smooth muscle cells, to engraft in humans, may necessitate arterial injury. Hematopoietic stem cells can be difficult to expand in culture and gene-modify, and very large numbers are required for engraftment in the absence of a toxic “conditioning” regimen. Lymphocytes possess a short lifespan, and human umbilical vein endothelial cells are limited in their use as autologous cells since they cannot be obtained from an adult.

In vivo delivery of Epo by the direct administration of replication defective viral vectors, such as adenovectors has already performed (Maione, D, et al., 2000, Human Gene Therapy, 11:859; Descamps, V et al., 1994, Human Gene Therapy, 5:979), and adeno-associated viral (AAV) vectors (Kessler, P. D. et al., 1996, Proc. Nat. Acad. Sci. USA, 91:11557) However, the utilization of Ad vectors and less so of MV vectors INCLUDE reports of immune response to AAV, is limited by their potential ability to elicit a host immune response.

Replication-defective retroviral vectors allow integration of the provirus into the host chromosomal DNA, ensuring high level, long-term transgene expression in target and progeny cells. Accordingly, although they cannot be directly injected in uninjured tissue due to their necessity of cell division for nuclear access, murine oncoretrovectors may be useful tools for ex vivo gene transfer into dividing cells that can proliferate ensuing transduction. Non-viral approaches for Epo delivery have been assayed through naked plasmid DNA injection and gene electrotransfer (Rizzuto, G. et al., 1999, Proc. Nat. Acad. Sci., USA, 96:6417). As compared to viral vectors, gene expression from plasmid DNA may be insufficient to provide therapeutic protein levels, especially in larger mammals. Extrapolating the requirements from a mouse to a human based on body weight, substantially high amounts of plasmid DNA would be needed to achieve a significant biological effect. A further disadvantage is that gene electrotransfer usually requires surgically exposing the target muscle tissue.

The novelty shown in the present study was to determine if gene-modified murine MSCs could engraft by intraperitoneal injection in mice, without requirement of conditioning immunosuppressive therapy such as chemotherapy or radiotherapy, and subsequently express sufficient levels of the gene product. It is shown that primary murine MSCs transduced with a retrovector containing murine Epo cDNA can be implanted by intraperitoneal administration in non-myeloablated, immunocompetent mice and secrete Epo in the systemic circulation. It is reported that the levels of Epo released in vivo are sufficient to cause a supraphysiological effect as evidenced by a significant and sustained enhancement of blood hematocrit which is dependent on the amount of implanted MSCs and on their ex vivo protein secretion levels. These data strongly support the use of transgenic autologous stroma for delivery of pharmacological levels of soluble plasma proteins.

Materials and Methods

Cell Culture of Murine Fribroblasts

GP+E86 ecotropic retrovirus-packaging cell line from American Type Culture Collection (ATCC) was cultured in Dulbecco's modified essential medium (DMEM) (Wisent Technologies, St.Bruno, QC) supplemented with 10% heat-inactivated fetal bovine serum (FBS) (Wisent) and 50 Units/ml penicillin, 50 □g/ml streptomycin (Pen/Step) (Wisent). National Institutes of Health (NIH) 3T3 mouse fibroblast cell line, obtained from ATCC, was grown in DMEM with 10% FBS and 50 Units/ml Pen/Step. All cells were maintained in a humidified incubator at 37° C. with 5% CO₂.

Generation of Retroviral Vector and of Virus-Producing Cells

The retroviral plasmid vector pIRES-EGFP, containing sequences derived from murine stem cell virus (MSCV) and from MFG, was previously generated (Galipeau, J. et al., 1999, Cancer Research, 59:2384). This construct comprises a multiple cloning site linked by an internal ribosomal entry site (IRES) to the enhanced green fluorescent protein (EGFP) (Clontech Laboratories, Palo Alto, Calif.). The retroviral vector pEpo-IRES-EGFP (FIG. 1) was synthesized by obtaining the cDNA for mouse erythropoietin by Bam H1 digest of a pBluescript-based construct graciously provided by Jean M. Heard (Institut Pasteur, Paris) and ligating it with a Bam H1 digest of pIRES-EGFP.

For the manufacture of recombinant virus-producing cells, the pEpo-IRES-EGFP construct (5 □g) was linearized by Fsp1 digest and co-transfected, utilizing lipofectamine reagent (Gibco-BRL, Gaithesburg, Md.), with 0.5 μg pJ6ΩBleo drug resistance plasmid (Morgenstem, J. P. et al., 1990, Nuc. Acid Res., 18:1068) generously given by Richard C. Mulligan (Children's Hospital, Mass.), into GP+E86 packaging cells. Stable transfectants were selected by 5-week exposure to 100 μg/ml zeocin (Invitrogen, San Diego, Calif.), thus giving rise to the polyclonal virus-producing cells GP+E86-Epo-IRES-EGFP. The control GP+E86-IRES-EGFP producers were generated in this same manner. GFP expression in cells was assessed by flow cytometry analysis utilizing an Epics XL/MCL Coulter analyzer and gating viable cells based on FSC/SSC profile. An additional population of GP+E86-Epo-IRES-EGFP producers was obtained following sorting of cells based on green fluorescence using a Becton Dickinson FACSTAR™ sorter. Retroparticles from all producers were devoid of replication competent retrovirus as was determined by GFP marker rescue assay employing conditioned supernatants from transduced target cells.

Titer Determination of Retrovirus Producers

To assess the titer of GP+E86-Epo-IRES-EGFP and GP+E86-IRES-EGFP producers, NIH 3T3 fibroblasts were seeded at a density of 2 to 4×10⁴ cells per well of 6-well tissue culture plates. The next day, cells were exposed to serial dilutions (0.01 μl to 100 μl) of 0.45 μm filtered retroviral supernatants, in a total volume of 1 ml complete media with 6 □g/ml lipofectamine. Cells from extra test wells were counted and averaged to disclose the baseline cell number at moment of virus addition. Three days later, the percentage of GFP-expressing cells was ascertained by flow cytometry analysis. The titer was calculated using the following equation by considering the virus dilution that yielded 10-40% GFP-positive cells. Titer (infectious particles/ml)=(% GFP-positive cells)×(amount of target cells at start of virus exposure)/(volume of virus in the 1 ml applied to cells).

Whole bone marrow was harvested from the femurs and tibias of 18-22 g female C57BI/6 mice (Charles River, Laprairie Co., QC) and plated in DMEM supplemented with 10% FBS and 50 Units/ml Pen/Step. After 4 to 5 days of incubation at 37° C. with 5% CO₂, the nonadherent hematopoietic cells were discarded and the adherent MSCs were gene-modified as follows. Media was removed from MSCs and replaced with 0.45 μm-filtered retroviral supernatant from subconfluent GP+E86-Epo-IRES-EGFP or control GP+E86-IRES-EGFP producers twice per day for three consecutive days in the presence of 6 μg/ml Lipofectamine™. The resulting genetically engineered stroma was subsequently expanded for 2-3 months. A second preparation of Epo-IRES-EGFP modified MSCs arose ensuing a 2 to 3 month expansion of cells transduced once per day for 6 successive days for each of 2 consecutive weeks with retroparticles from subconfluent sorted GP+E86-Epo-IRES-EGFP cells, with 6 μg/ml Lipofectamine™. GFP expression in gene-modified stroma was evaluated by flow cytometry analysis to allow an estimate of the gene transfer efficiency. Supernatant was collected from genetically engineered cells and Epo secretion was assessed by photometric enzyme-linked immunosorbent assay (ELISA) specific for human Epo (Roche Diagnostics, Indianapolis, Ind.). Animals were handled under the guidelines promulgated by the Canadian Council on Animal Care.

Stroma Implantation and Blood Sample Analysis

Epo-IRES-EGFP as well as IRES-EGFP genetically engineered MSCs were trypsinized, concentrated by centrifugation, and 10⁷ cells suspended in 1 ml of serum-free RPMI media (Wisent) implanted by intraperitoneal injection into each of 3 to 5 syngeneic mice. In an additional experiment, the second preparation of Epo-IRES-EGFP modified stromal cells at the various concentrations of 10⁵, 10⁶, 5×10⁶ and 10⁷ cells in 1ml of media was injected into the peritoneum of 4 cohorts of 3 to 4 syngeneic C57BI/6 mice. Mice that received marrow stroma transduced with IRES-EGFP retroparticles were referred to as “Control mice” whereas those that were implanted with Epo-IRES-EGFP modified stroma constituted “Epo mice”. Blood samples were collected from the saphenous vein with heparinized micro-hematocrit tubes (Fisher Scientific, Pittsburgh, Pa.) prior to and every ˜1 to 4 weeks post-implantation. Mice were monitored for −8 months. Hematocrit levels and plasma Epo concentrations were ascertained from blood samples. Specifically, hematocrits were quantitated by standard microhematocrit procedure, and Epo concentrations in plasma preparations were assessed by ELISA for human Epo (Roche Diagnostics).

Southern Blot Analysis

Genomic DNA was isolated from Epo-IRES-EGFP stably transduced primary murine MSCs, as well as from unmodified marrow stroma, utilizing the QIAamp™ DNA mini kit (Qiagen, Mississauga, ONT). For Southern blot analysis, 10 □g of genomic DNA was digested with EcoRV, separated by electrophoresis in 1% agarose, and transferred to a Hybond-N™ nylon membrane (Amersham, Oakville, ONT). The probe was prepared by ³²P radiolabeling of the EGFP complete cDNA utilizing a Random Primed DNA Labeling Kit (Roche Diagnostics) and was hybridized with the membrane. The blot was washed, irradiated, and exposed to Kodak X-Omat™ film.

Results

GFP Expression and Titer of Retrovirus Producers

To determine gene transfer efficiency and transgene expression in stably transfected cells, flow cytometry analysis for GFP expression was performed. The proportion of GFP positive cells in the polyclonal producer populations GP+E86-Epo-IRES-EGFP, and GP+E86-Epo-IRES-EGFP sorted based on green fluorescence, were 34% and 97%, respectively, as compared to under 3% for parental untransfected cells. To evaluate the quantity of infections particles released by these producers, a titration assay using their retroviral supernatant was conducted and the viral titers obtained were ˜2.4×10⁵ and ˜4.0×10⁵ infections particles per ml, respectively.

GFP Expression and Epo Secretion by Gene-Modified Marrow Stroma

In order to ascertain the degree of transgene expression in genetically engineered murine marrow stroma, flow cytrometry analysis for GFP expression was carried out. The proportion of GFP-positive cells was 54% for Epo-IRES-EGFP transduced. stroma, and 91% for the 2^{nd} preparation of Epo-IRES-EGFP modified MSCs. To establish that murine MSCs transduced with Epo-IRES-EGFP secrete Epo in vitro, and quantitate the level, supernatant collected from these cells was analyzed by enzyme-linked immunosorbent assay (ELISA) for human Epo. The first and second generation of Epo-IRES-EGFP modified stroma was thus revealed to secrete 1.7 and 17 Units of Epo per 10⁶ cells per 24 hours, respectively. There was no Epo detected in the supernatant collected from control IRES-EGFP transduced MSCs.

Southern Blot Analysis

To ascertain that the recombinant retroviral construct Epo-IRES-EGFP did not undergo rearrangements or deletions prior to its integration as proviral DNA in the genome of transduced MSCs, Southern blot analysis was conducted. A probe complementary to the GFP reporter allowed the detection of a DNA band consistent with the 3436bp fragment anticipated from EcoRV digest of integrated unrearranged Epo-IRES-EGFP proviral DNA (FIG. 1). No subgenomic retrovector integrant was detected.

Hematocrit of Mice Implanted with Gene-Modified Stroma

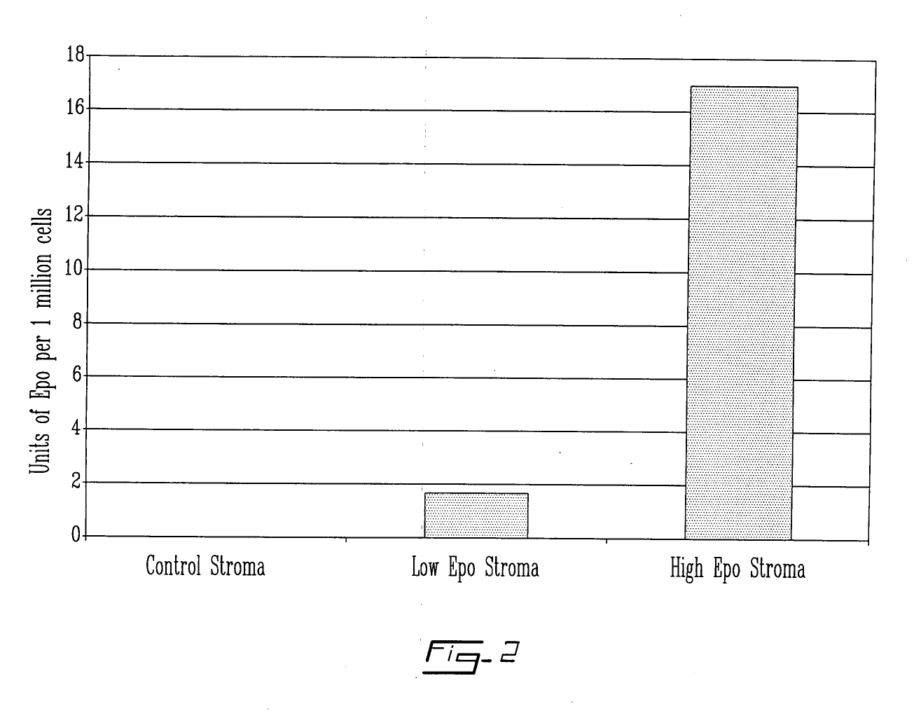

To determine if Epo secretion from Epo-IRES-EGFP transduced stroma implanted by intraperitoneal injection in non-myeloablated, immunocompetent mice can lead to a measurable effect, the hematocrit was measured prior to and up to ˜8 months post-implantation. The hematocrit of C57BI/6 mice implanted with 10⁷ Epo-IRES-EGFP modified MSCs secreting 1.7 Units of Epo per 10⁶ cells per 24 hours, increased from a basal level of 53±1.2% (mean±SEM) to 76±0.9% within 2 weeks following implantation (FIG. 2). The hematocrit of these recipients continued to rise further, reaching a value of 88±0.9% at 12 weeks and thereafter slowly declined but remained at hematocrit levels of greater than 70% until 28 weeks post-implantation. At 35 weeks following stroma administration, the hematocrit of mice had decreased to 57±6.5%. A parallel group of mice received 10⁷ IRES-EGFP transduced MSCs. These control mice maintained hematocrit levels ranging between 51 and 57% throughout this study.

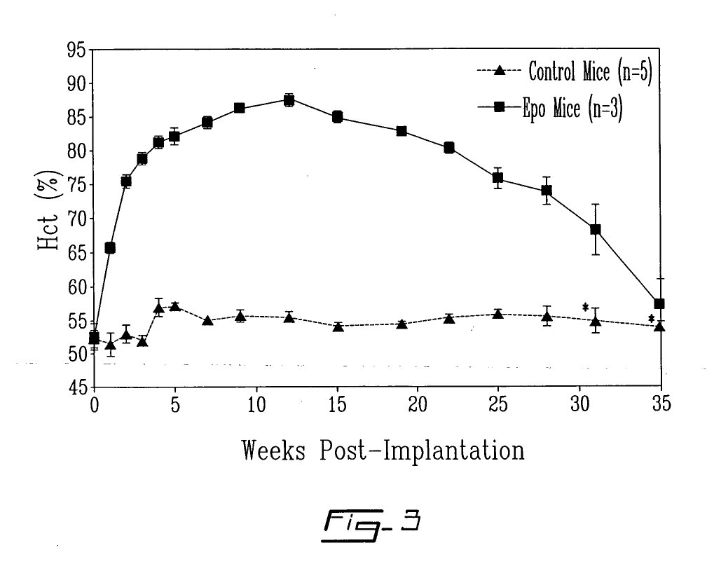

In order to establish if there is a dose-response relationship between the number of Epo-IRES-EGFP modified stromal cells injected and the resulting hematocrit, the following investigation was performed. Cohorts of mice were implanted with either 10⁵, 10⁶, 5×10⁶ or 10⁷ of Epo-IRES-EGFP engineered MSCs noted to secrete in vitro 17 Units of Epo per 10⁶ cells per 24 hours. Peripheral blood was collected and hematocrit measured over time as shown in FIG. 3.

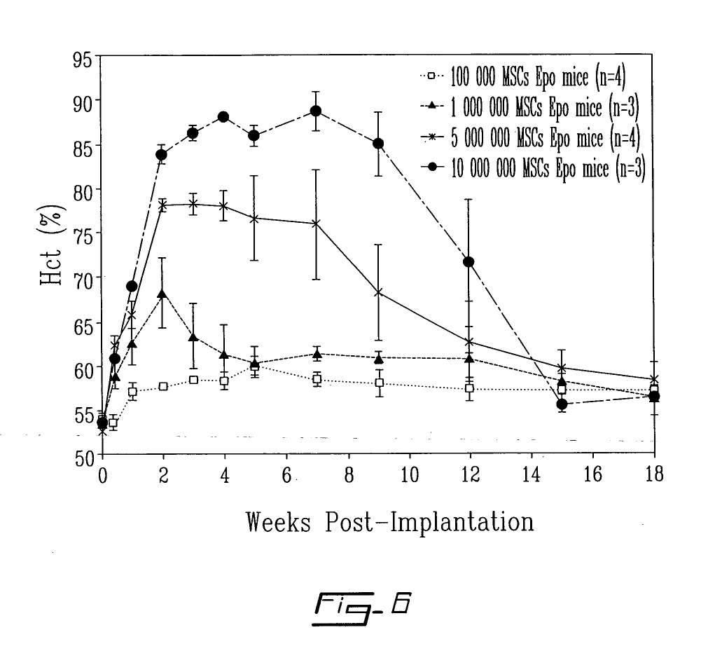

The hematocrit of mice that received 10⁵ Epo-secreting stromal cells slightly increased to a peak value of 60±1.1% at 5 weeks post-implantation. In mice injected with 10⁶ Epo-IRES-EGFP transduced MSCs, blood hematocrit rose to maximum of 68±3.8% at 2 weeks succeeding implantation and then quickly declined to a steady ˜61% observed until week 12. The recipients of 5×10⁶ Epo secreting MSCs had an increase in hematocrit that attained a value of ˜78% at 2 weeks post-implantation, remaining above 75% until 7 weeks following stroma administration. Moreover, the hematocrit of mice implanted with 10⁷ of these gene-modified MSCs (secreting 17 Units of Epo per 10⁶ cells per 24 hrs) attained the highest level at 4 weeks (˜88%) (FIG. 6), thenceforth persisting at ˜85% or greater up to week 9 and over 70% up to week 12.

Epo Concentration in Blood Plasma of Mice Implanted with Gene-Modified Marrow Stroma

To quantify the plasma concentration of Epo in mice administered Epo-IRES-EGFP engineered marrow stroma, plasma from harvested blood was analyzed by Epo ELISA. As done by others in the field, ELISA kits for detection of human Epo are utilized to detect mouse Epo.

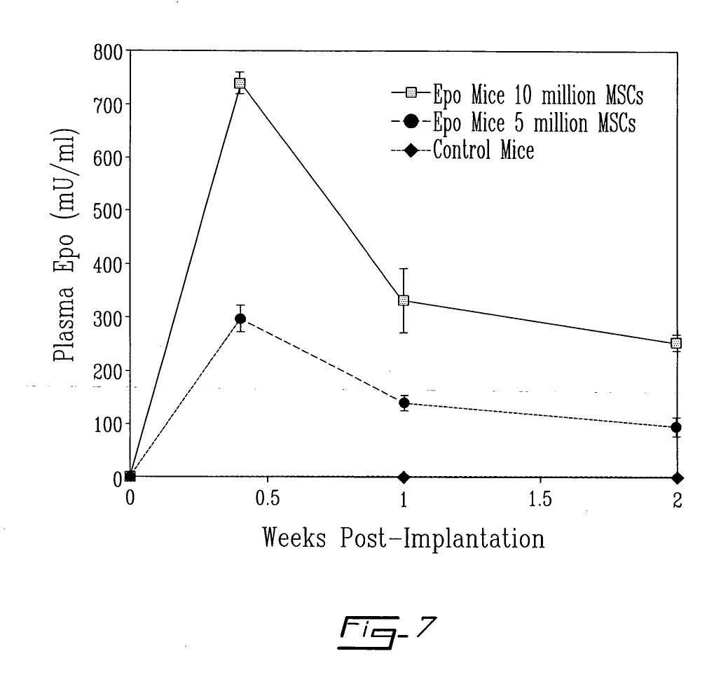

Epo levels detected in the plasma of mice implanted with 10⁷ gene-modified MSCs secreting in vitro 1.7 Units of Epo per 10⁶ cells per 24 hours, rose from a pre-implantation value of ˜50 mUnits/ml to 270±41, 264±62, and 199±38 mUnits/ml at 1, 2, and 3 weeks ensuing stroma administration, respectively (FIG. 7). Moreover, the concentration of Epo measured in plasma collected at 7 weeks and longer following implantation was below 10 mUnits/ml.

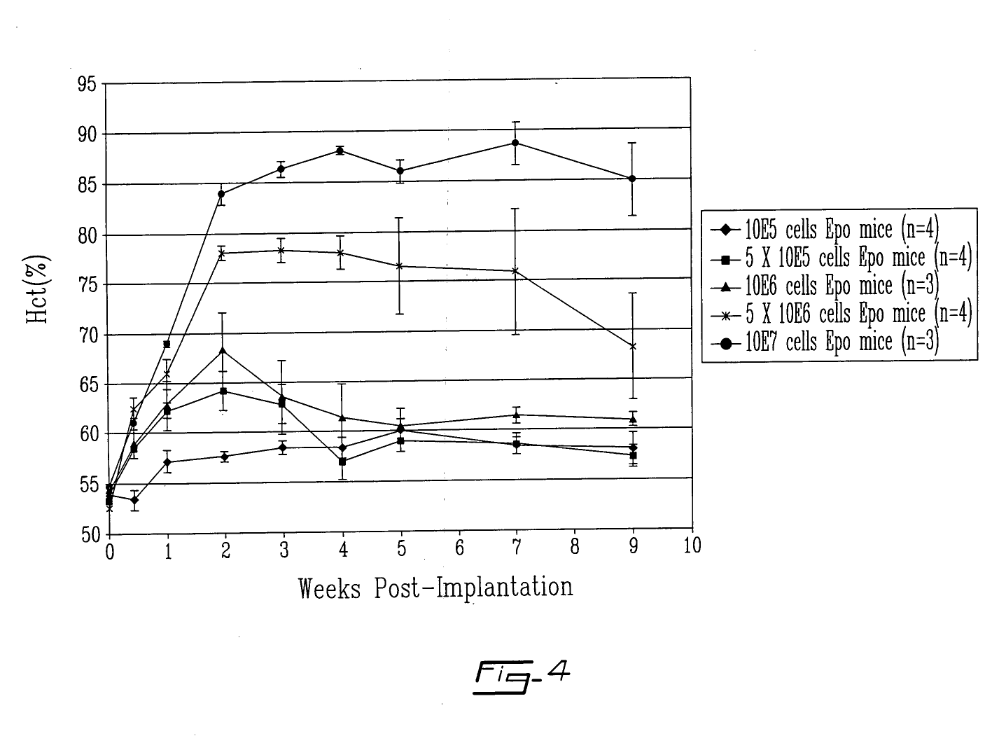

Mice that received 10⁷ and 5×10⁶ Epo-IRES-EGFP engineered syngenic MSCs secreting in vitro 17 Units of Epo per 10⁶ cells per 24 hours, exhibited a rise in plasma Epo concentration to 740±20 and 298±25 mUnits/ml, respectively, at 3 days post-implantation (FIG. 4), which declined proportionally by over 50% to 333±60 and 141±15 mUnits/ml, respectively, at 1 week, and by over 65% to 255±15 and 96±18 mUnits/ml, respectively, at 2 weeks. The concentration of Epo detected in the plasma of these mice at 7 weeks or greater post-implantation was under 20 mUnits/ml.

### Conclusion

The present experiment represents a novel demonstration of systemic secretion of supraphysiological quantities of a soluble gene product from genetically engineered syngeneic murine MSCs implanted by intraperitoneal injection in non myeloablated, immunocompetent mice.

As illustrated in FIGS. 3 and 4, a correlation between the number of Epo gene-modified MSCs implanted in mice and the degree of plasma Epo elevation and of consequent hematocrit augmentation was noted. As was similarly observed with Epo-secreting skin fibroblasts, the present findings indicate that desired levels of protein delivery and thus therapeutic effect can be modulated by varying the amount of gene-modified MSCs implanted, taking into account their in vitro protein secretion levels. The present results therefore reveal that in vitro secretion levels of transgene product can somewhat predict systemic protein delivery in vivo and thence the amount of gene-modified MSCs that must be implanted i.p. to achieve the preferable levels of recombinant protein in the recipient.

In the present experiment, a cell dose approximately 200×10⁶ cells/kg (or 5×10⁶ cells per 25 g mouse) has been found to lead to supraphysiological production of Epo. Therefore, human MSCs secreting comparable amounts of hEpo may have a similar effect, and that cell dose required for an average 70 kg adult would be clinically realizable. In light of this strong dose effect relationship of Epo secretion and hematocrit, a smaller dose of MSCs secreting higher levels could be used.

Another important asset of this cell therapy approach is that autologous gene-modified and tissue culture expanded MSCs can be cryopreserved which would allow their reimplantation if so later thereafter required.

In conclusion, the present data validate the utility of using gene-modified autologous bone marrow stroma as a vehicle for sustained systemic production of recombinant therapeutic proteins in immunocompetent recipients and without the major drawback of myeloablation. This example provides a clear demonstration for applications of MSCs as safe and delivery vehicles of beneficial gene products in the treatment of a large spectrum of inherited or acquired serum protein deficiencies. Possible corrective proteins may include growth hormone, clotting factors, cytokines such as granulocyte colony stimulating factor, enzymes such as glucocerebrosidase, antineoplastic proteins, and anti-infection agents.

### EXAMPLE V

### Therapeutic Angiogenesis by Autologous Stromal Cells

Materials and Methods

Harvest, Culture and Retroviral Transduction of Rodent MSCs

Bone marrow are harvested from C57BI/6 female mice, weight=16-18 gm (Charles River Laboratory, Laprairie Company, PQ). The mice are sacrificed by CO₂ asphyxiation method. Immediately after sacrificing the mouse, the femoral and tibial bones are collected from both hind limbs, taking care to avoid injuring the bones. Both ends of the bones are to be cut away from the diaphyses with scissors. The bone marrow plugs are hydrostatically expelled from the bones by insertion of 25-gauge needles fastened to 10 ml syringe filled with complete medium. Medium: Dulbecco's Modified Eagle's Medium (DMEM™) containing 10% fetal bovine serum and antibiotics (50 U/ml Penicillin G and 50 μg/ml Streptomycin from Wisent Inc.). Bone marrow cells are plated on tissue culture dishes in the same medium. The culture dishes are incubated at 37° C. with 5% CO₂. The non-adherent hematopoietic cells are discarded five days later and media are replaced once per week. To prevent the stromal cells from differentiating or slowing their rate of division, each primary culture is replated (first passage) to two new 10 cm plates when the cell density within colonies becomes 80% to 90% confluent approximately 2 weeks after seeding or sometimes even before. Trypsin 0.05% is used for releasing the cells from the plate.

Marrow Stromal Cell (MSC) Retroviral Labelling

All gene transfer are performed utilizing replication-defective retroviral vectors. There is much background and technical information regarding their use in the appended materials. In brief, an implant genetic-labelling vector that encode for prokaryotic β-galactosidase has been developed. This has been successfully utilized to label rodent stromal cells as detailed in the data section. Labelled MSCs and their differentiated progeny can be tracked post-implantation by histochemical X-gal stain performed on frozen sections derived from implanted tissues and Matrigel™. This assay will allow us to distinguish the implanted MSCs (and their differentiated progeny) from endogenous (non-MSC) cells recruited in to the neoangiogenic process.

Cultured MSCs are trypsinized with 0.05% Trypsin+0.53 mM EDTA and replated. The next day, they are transduced with LacZ retroviral particles once per day for three consecutive days with Lipofectamine™ Reagent “Life Technologies” (3 μL of Lipofectamine™ 2 mg/ml solution for each 1 ml of virus medium). At each transduction, the marrow stromal cells medium is replaced with the supernatant from the LacZ-GP+E86 cells (after being filtered through Millex®-HV 0.45 μm filter). Five days after the last transduction, a stromal cells culture plate are selected for histochemical staining for β-galactosidase activity to determine percentage of cells expressing β-galactosidase. The cells are fixed in 1% glutaraldehyde for 5 minutes at room temperature, then the cells are washed with phosphate buffered saline. Staining solution (500 μL) are added which contains 1 mg/ml 5-bromo-4-chloro-3-indoyl-β-D-galactoside (X-gal), 1 mM EGTA, 5 mM K₃Fe(CN)₆, 5 mM K4Fe(CN)₆.3H₂O, 2 mM magnesium chloride, and 0.01% sodium deoxycholate. Then cells are incubated at 37° C. protected from light for 16 hours.

Transduction of MSCs with Retrovector Encoding for mEPO

A bicistronic retroviral vector encoding for mEPO and GFP was developed. Related control vectors expressing GFP only have also been synthesized and tested. The cDNA for rat erythropoietin (rEPO) was linked and retrovectors encoding for its production were generated. The purpose of which is to facilitate histochemical tracking (by X-gal staining) of EPO secreting MSCs in vivo.

For gene transfer into mouse stroma, ecotropic retroparticles derived from the GP+E86 retroviral packaging cell line was used. For gene transfer into rat stroma, amphotropic GP+Am12 retroviral packaging cell line was used. In brief, all retroparticles contain a replication-defective retrovirus carrying the murine EPO gene and the reporter gene Green fluorescent protein (GFP). The EPO gene cDNA is inserted upstream of an IRES (Internal Ribosomal Entry Site), and both the EPO cDNA and the GFP reporter gene are expressed in transduced cells by means of LTR (Long Terminal Repeat) promoter element. A control retrovirus carries only the reporter gene GFP downstream of IRES, and will act as a negative control. Retroviral transduction of stromal cells is done two weeks after transducing the stromal cells with B-galactosidase retrovector. Once the stromal cells have recovered, half the culture plates are transduced with EPO retrovector and the other half are transduced with control GFP retrovector. Transduction is done once per day for 6 consecutive days for each of 2 weeks (with Lipofectamine™ as described above). The genetically engineered MSCs are allowed to expand in culture for over 4 weeks. Transduction efficiency is measured by determining the percentage of cells expressing the GFP reporter gene (as a reflection of EPO expressing cells) using flow cytometry analysis.

Matrigel™ Assay

There are many in vitro and in vivo assays to ascertain angiogenic (and anti-angiogenic) activity of drugs and other compounds. An in vivo assay was elected, where implanted MSCs could be analyzed functionally and histologically and that would most closely recapitulate physiological angiogenesis. For these reasons, the implanted Matrige™ (obtained from Becton Dickinson Canada Inc.) assay has been established. Matrigel™ Matrix is a reconstituted basement membrane isolated from the EHS (Engelbreth-Holm-Swarm) mouse sarcoma, a tumor rich in extracellular matrix proteins. It is composed of laminin, collagen IV, entactin, heparan sulfate proteoglycan, matrix metalloproteinases, growth factors, and other undefined components. It is also available in modified preparation “Growth Factors Reduced” (GFR) developed by Taub et al. (Proc. Natl. Acad. Sci. USA (1990) vol. 87:4002-4006). It closely mimics the structure, composition, physical properties, and functional characteristics of the basement membrane in vivo. It basically has a similar chemical structure to the basement membrane. It exists as a semi-liquid at 4° C. and rapidly becomes solid at 22-35° C. As shown in preliminary data, genetically-engineered MSCs can be suspended in Matrigel™, implanted subcutaneously in mice and subsequently retrieved for phenotypic analysis. Matrigel™ is widely used in vitro and in vivo experiments because it has the following attractive features: I) it forms a three dimensional model to study cells behavior and differentiation. Quantitative and qualitative assays including histological and immunohistochemical studies can be easily used with this model; II) it can act as a reservoir for growth factors, or reagents under study giving a sustained and slow release into surrounding media; and III) it allows and supports cell survival, proliferation and differentiation into different structures. It provides a physiologically relevant environment for studies of cell morphology, biochemical function, migration or invasion, and gene expression.

C57BI/6 female mice (Charles River Laboratory, Laprairie Company, PQ) are used for experimental purposes. These inbred strains of mice are used as donors and recipients of MSCs to simulate autologous implant clinically. All animals are studied and handled as per the guidelines of the Canadian Council on Animal Care “Guide to the Care and Use of Experimental Animals”.

Matrigel™ Implantation and Retrieval

It has been observed that up to 4×10⁶ MSCs can be resuspended in 1 ml of Matrigel™, in liquid form at 4° C. A volume of 0.5 ml of this mixture can be implanted subcutaneously in a C57bl mouse and will form a Matrigel™ bed. Two weeks following implantation, mice are sacrificed and Matrigel™ plug excised and handled for histochemical analysis. The abdominal wall skin is opened in the midline. With gentle dissection, the Matrigel™ plug is removed, taking care to avoid puncturing or dividing the Matrigel™. Each plug is divided into two parts. One part is fixed in 10% buffered formalin, and embedded in paraffin to be sectioned and stained with hematoxylin and eosin for light microscopy study. The other part is embedded in OCT compound, snap-frozen in liquid nitrogen, and cut into 51 μm thick sections.

Results

MSCs can be implanted in different organ compartments such as brain, muscle and heart without requiring ablation therapy; (ii) MSCs genetically-engineered to secrete EPO can be implanted in animals and lead to biologically-verifiable effects; and, (iii) MSCs can promote and directly participate in a neo-angiogenic process in vivo.

Genetic Engineering of Rodent MSCs and Organ Implantation

Series of retroviral vectors that express the Green Fluorescent Protein (GFP) reporter have been designed and their utility was examined for genetic engineering of rat MSCs. In other series of experiments, rat stromal cells were retrovirally engineered to express either GFP or the bacterial beta-galactosidase reporter gene.

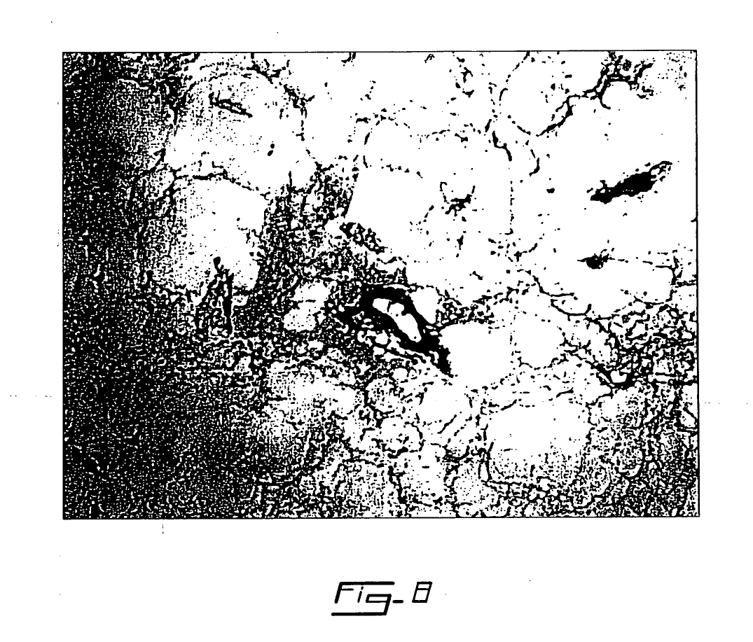

In related work, it has been tested whether stromal cells can engraft in myocardium, this towards development of cell therapy for heart disease. It was shown that DAPI-labelled rat stroma engrafts and persists in heart muscle. Stroma was also implanted in brain and muscle. Two weeks following implantation of 100,000 stromal cells in brain parenchyma, animals were sacrificed and sections obtained from whole brain mounts. At the same time, 1,000,000 stromal cells were implanted intramuscularly and muscle sections taken at time of sacrifice (FIG. 8). Live beta-galactosidase expressing stromal cells are clearly recognized. These data strongly demonstrate that tissue-implanted stromal cells can engraft locally at injection site without need of “conditioning” immunosuppressive regimen such as radiotherapy.

In Vivo Implantation of Mouse MSCs Engineered to Secrete EPO

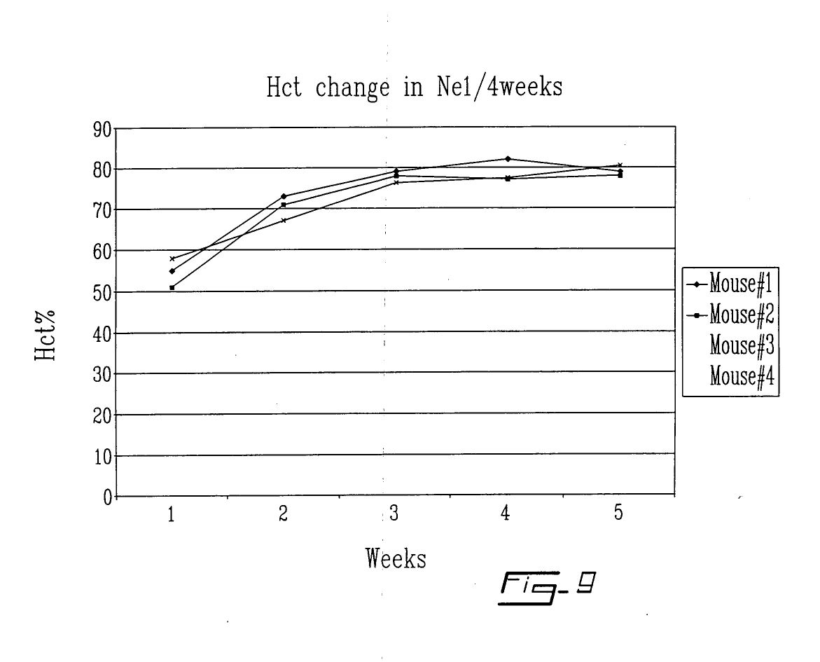

The mouse EPO (mEPO) cDNA has been cloned into a bicistronic retroviral vector comprising the green fluorescent protein (GFP) reporter gene downstream of an internal ribosome entry site (IRES). The resulting construct was stably transfected into GP+E86 packaging cells, consequently generating Epo-GP+E86 cells producing ˜2.5×10⁵ infectious particles per ml, as determined by titer assay on NIH 3T3 cells. Primary bone marrow stromal cells from C57BI/6 mice were transduced with retroparticles from Epo-GP+E86 cells once a day for 3 consecutive days and subsequently allowed to expand in culture for ˜2 months. These genetically engineered cells were revealed to secrete ˜200 mU of Epo per 10⁶ cells per 24 hours, as determined by enzyme-linked immunosorbent assay (ELISA). In addition, 54% of this Epo-transduced stromal cell population expressed GFP, as ascertained by flow cytometry analysis. Provirus integration and lack of rearrangement in transduced cells was confirmed by Southern Blot analysis of restriction enzyme digested genomic DNA. Three test isogenic mice had 10⁷ Epo-secreting marrow stromal cells implanted into their abdominal cavity by intraperitoneal (i.p.) injection. The hematocrit of these recipients rose from a basal level of 53±2% (mean±S.E.M.) to 76±1% within two weeks following implantation and persisted to escalate further attaining a value of 88±1% at 12 weeks post-implantation. A parallel cohort of animals (n=5) received 10⁷ stromal cells engineered with a control retrovector. Their hematocrit remained at basal levels (51 to 57%) throughout the study. (FIG. 9). These findings strongly support the use of bone marrow stroma as a delivery vehicle for sustained systemic production of recombinant therapeutic proteins in normal immunocompetent animals.

MSCs and Angiogenesis In Vivo

In another experiment using the Matrigel™ Angiogenesis assay described in the proposal, 0.5 ml of Matrigel™ mixed with 1.0×10⁶ marrow stroma cells that were genetically modified to express the reporter GFP protein was implanted into C57BI/6 mice subcutaneously. Other groups of mice were injected subcutaneously with plain Matrigel™ as a negative control. The implants were retrieved after two weeks. It has been found that the plain Matrigel™ implants did not elicit any visible tissue reaction or neo-angiogenesis and they were clear and transparent at time of retrieval. On the other hand, there were macroscopically visible new blood vessels that grew into the Matrigel™ implants containing marrow stromal cells. The microscopic images confirm the macroscopic findings. A further experiment was performed where β-galactosidase-expressing stromal cells were matrigel embedded. As shown in FIG. 8, a cross-sectioned blood vessel is clearly composed of X-gal-staining endothelial cells. These data generated with this model strongly demonstrate that marrow stromal cells can actively induce and participate in the generation of new blood vessels.

Erythropoietin Secreting Stroma and Angiogenesis In Vivo

It is also shown that stroma secreting EPO enhances the stroma-associated angiogenic effect.

### EXAMPLE VI

### Bone Marrow Stromal Cells Elicit a Potent VEGF-Dependent Neo-Angiogenic Response In Vivo

Materials and Methods

Animals

Female C57BI/6 mice (18-20 gm) obtained from Charles River Laboratories (Laprairie Co., Quebec) were used. These isogenic mice were used as donors and recipients of MSC to simulate autologous implantation. All animals were studied using guidelines published in “The 1996 NIH Guide: Guide for the Care and Use of Laboratory Animals 7^{th} Edition” and the uGuide to the Care and Use of Experimental Animals” of the Canadian Council on Animal Care”.

Harvest and Culture Expansion of Bone Marrow Stromal Cells

Female C57BI/6 mice were sacrified and bone marrow cells harvested by flushing femurs and tibias with DMEM supplemented with 10% FBS and 50 U/ml Penicillin/Streptomycin. Whole marrow was plated in tissue culture dishes and 5-7 days later discarded the non-adherent hematopoietic cells and maintained the adherent bone marrow stromal cells at 37° C. with 5% CO₂. Culture expandedMSCs was done for 4-5 months.

Generation of LacZ Gene-Modified Marrow Stromal Cells

Retrovirus-producing cells were generated by transfecting or transducing packaging cell lines GP+E86 and GP+Am12 with retroviral constructs containing as a selectable marker the green fluorescent protein (GFP) gene or the drug resistance gene human cytidine deaminase (hCD). Filtered viral supernatants to transduce primary murine MSCs were used, and assessed GFP transgene expression by flow cytometry analysis, as well as in vitro selective expansion of hCD engineered stroma using cytosine arabinoside (Ara-C). Both preparations of gene-modified stromal cells 75-95% beta-galactosidase were rendered expressing through exposure 1-2 times per day for 3-6 consecutive days (with 6 μg/ml lipofectamine) to filtered supernatant from GP+E86 cells producing LacZ gene-containing retroparticles. The resulting groups of LacZ stromal cells was expanded for about an additional month before implantation in syngeneic mice. It has been possible to monitor and identify the implanted MSCs and their progeny in all sections by retroviral gene marking of MSCs with LacZ gene. This reporter gene encodes for a prokaryotic nuclear localized β-galactosidase enzyme, which gives a characteristic indigo-blue (in H&E stained sections) or green-blue colour (in sections stained with DAB) when incubated with X-gal solution.

Murine Matrigel™ Assay

Matrigel™ (Becton Dickinson, Bedford, Mass.) was used as a three dimensional in vivo model of angiogenesis. On the day of implantation, MSCs were trypsinized and counted. The following numbers of MSCs were used: Non-LacZ MSCs 2.0×10⁶ cells/ml of Matrigel™ (n=4), and LacZ MSCs 1.0×10⁶ (n=4), 2.0×10⁶ (n=8 mice for 14 days and another 8 mice for 28 days), 4.0×10⁶ (n=4) and 8.0×10⁶ MSCs/ml of Matrigel™ (n=4). MSCs were suspended in 50 μL of RPMI medium and then mixed the cells with 0.5 ml of Matrigel™. All the steps involving the Matrigel™ were done at 4° C. Matrigel™ was injected subcutaneously into the right flank of the mice using 25-guage hypodermic needles. At body temperature, Matrigel™ rapidly forms a semi-solid pellet. Either 500 ng of bovine bFGF (from R&D Systems, Minneapolis, Minn.) or 25 ng of murine VEGF 165 (Research Diagnostics Inc, Flanders, N.J.) was mixed with 0.5 ml of Matrigel™ per mouse (final concentration 1000 ng/ml for bFGF and 50 ng/ml for VEGF) which we implanted into a 14 days groups (n=4 mice for bFGF and n=4 mice for VEGF) and a 28 days group (n=4 for bFGF and n=4 for VEGF). As a negative control, 0.5 ml of plain Matrigel™ mixed with 50 μL of RPMI medium per mouse (n=4 mice for 14 days and n=4 mice for 28 days) was used. In addition, Matrigel™ containing 2.0×10⁶ LacZ-MSCs/ml mixed with either 4 μg/ml of rabbit polyclonal anti-murine VEGF neutralizing antibodies (n=5) or 4 μg/ml of non-specific rabbit polyclonal IgG antibodies (n=5) as a control was implanted for the effect of adding immunoglobulins to the MSCs (both antibodies from Peprotech, Rocky Hill, N.J.).

Matrigel™ Retrieval and Processing

At 14 or 28 days, mice were sacrificed using CO₂ asphyxiation. Rapidly, the chest opened and transfected the right atrial appendage. We inserted 25-gauge needle connected to 20 ml syringe filled with cold (4° C.) phosphate buffered solution into the left ventricle and infused about 15 ml into the systemic circulation of the mice followed by 15 ml of cold (4° C.) 2% paraformaldehyde (PFA). Then, a midline abdominal skin incision was opened and gently dissected a right-sided abdominal skin flap. The gel plug was carefully removed from the surrounding tissues and placed it in 2% PFA at 4° C. After 24 hours, we placed the gel plug in X-gal staining solution which consisted of 5 mM K₃Fe(CN)₆, 5 mM K₄Fe(CN)₆.3H₂O, 0.01% sodium deoxycholate, 2 mM MgCl₂, 1 mM EGTA, and 1 mg/ml X-gal made in wash solution (PBS with 0.02% NP40). After 16 hours, the specimens was fixed in 10% buffered formalin and embedded them in paraffin. Sections were cuted at 3-4 μm. From each specimen, we used the fifth and tenth sections for hematoxylin and eosin (H&E) staining and the sixth and seventh sections for immunohistochemical staining for PECAM-1 (CD31) and VEGF, respectively. The specificity of the blue staining produced by the X-gal was confirmed in vitro and in vivo. Non-specific staining was never seen in any Matrigel™ specimen not containing LacZ labelled MSC

Immunohistochemical, and Trichrome Staining

Sections were deparaffinized in toluene (5 minutes ×3) followed by rehydration in 100%, 95%, and 70% ethanol then tap water (5 minutes ×1 each). Antigen retrieval by heating the slides in 0.21% citric acid for 10 minutes was performed. The slides were washed in PBS (5 minutes ×3), followed by 10 minutes incubation in 3% hydrogen peroxide in methanol for blocking the endogenous peroxidase activity. Serum blocking was done by incubating the slides for 30 minutes in 5% bovine serum albumin (BSA) +5% normal donkey serum (NDS) diluted in PBS for CD31 sections, or 5% BSA +5% normal goat serum diluted in PBS for VEGF sections. Sections were incubated with the primary antibody (either polyclonal goat IgG anti-mouse CD31 (1:100), or polyclonal rabbit anti-VEGF (1:100) which recognizes the 165, 189 and 121 splice variants of VEGF, both from Santa Cruz Biotechnology, Santa Cruz, Calif.) diluted in the blocking solution for 1 hour at room temperature. Following several washes in PBS, sections were incubated for 30 minutes with the biotinylated secondary antibody (either donkey anti-goat IgG from Santa Cruz at 1:100, or goat anti-rabbit at 1:200 from BD Pharmingen, San Diego, Calif.). After washing in PBS (5 minutes ×3), an avidin-biotinylated horseradish peroxidase complex (Vectastain Elite ABC kit, Vector, Burlingame, Calif.) was used to detect the antibody complex followed by the peroxidase substrate DAB™ (DAB kit from Vector) which produces a brown stain. All sections were counterstained with Harris Hematoxylin and mounted them using flouromount. Every time immuno-staining was done, a corresponding negative control was included where all the steps were performed except the incubation with the primary antibody, and any non-specific staining was found with above technique. Modified Masson's trichrome staining was done.

In Vitro Differentiation of MSCs and Capillary Tube Assay

Two 30 mm culture plates were coated with Matrigel™ according to the manufacturer's instructions. MSCs were seeded on the Matrigel™ at 2×10⁴ cells per plate in DMEM with 10% FBS and incubated at 37° C. with 5% CO₂. In one of the two plates, murine VEGF 165 was added to the Matrigel™ and the medium at 50 ng/ml concentration. After 24 hours, MSCs with VEGF started to arrange forming tubes that became more mature and vascular like structures formed of more than one layer of cells over the next few days. The tube formation was observed using an inverted phase contrast microscope (Axiovert 25™, Carl-Zeiss, North York, Ontario) and images were captured using Contax 167MT™ camera (Kyocera Corp., Tokyo, Japan). MSCs were cultured in 6-well plates over cover slips in the same medium described above with and without murine VEGF 50 ng/ml for 14 days. Immunoflourescence staining was performed on these cells after fixation in ice-cold methanol for 20 minutes at −20° C. followed by serum blocking in 5% BSA and 5% NDS in PBS for 30 minutes. Cells (except negative controls) were incubated with goat anti-mouse CD31 for 1 hour at room temperature. After several rinses in PBS, cells were incubated with donkey anti-goat IgG antibody for 30 minutes at room temperature. Cells were washed with PBS then incubated with streptavidin-Texas red (1:500) for 30 minutes, and then washed several times with PBS. Cover slips were mounted on slides with Gelvatol™.

Microscopy and Vascular Density

All sections were examined with an Olympus BX60 microscope. Digital images were transferred to a computer equipped with Image Pro™ software (Media Cybernetics, Baltimore, Md.). In H&E stained sections, only tubular structures were considered as blood vessels within the Matrigel™ that were lined with endothelium and had patent lumen containing erythrocytes (although the number of erythrocytes was markedly reduced in large blood vessels due to the fixation by perfusion). In sections stained with anti-CD31 antibody, only tubular structures we considered as blood vessels within the Matrigel™ that were CD31 +. For vascular density measurements, the surface area of each section (excluding the capsule) was measured using 400×magnification and Image Pro™ software and blood vessels were counted in each field as was measured the area. The vascular density was expressed as blood vessels (BV)/mm². Diameter of blood vessels was measured using the same software.

Statistical Analysis

All data are expressed as the mean±SD. All statistical analysis were carried using the SPSS version 10.0 software for Windows (SPSS Inc., Chicago, Ill.). A P-value of less than 0.05 was considered as statistically significant. Student's t-test was used to compare the mean vascular density at 14 and 28 days. Analysis of Variances (ANOVA) was used to do all the other groups of comparisons followed by Scheffe's multiple comparison test.

Results

Marrow Stromal Cells (MSCs)

When whole marrow aspirates are placed in culture, two populations distinguish themselves promptly: (i) “adherent” fibroblast-like cells and (ii) a mixture of “free-floating” hematopoietic cells. The fibroblast-like cells will give rise to colonies also known as Colony Forming Units-Fibroblast (CFU-F), hereafter referred to as Marrow Stromal-Cells (MSCs). In vitro and in vivo studies showed that MSCs are pleuripotent and have the ability to differentiate into several cell types including osteoblasts, chondroblasts, fibroblasts, adipocytes, skeletal myoblasts and cardiomyocytes. In addition to their stem cell ability, these cells are abundant in all age groups, easy to harvest, culture and expand in vitro which identify them as a desirable cell type for autologous cell therapy. In this experiment, the utilization of the MSCs was explored for the production of new blood vessels in mice where their ability to stimulate angiogenesis and arteriogenesis and to differentiate into endothelial cells participating in the newly formed vascular structures (i.e. vasculogenesis) was assessed.

MSCs Stimulate Angiogenesis

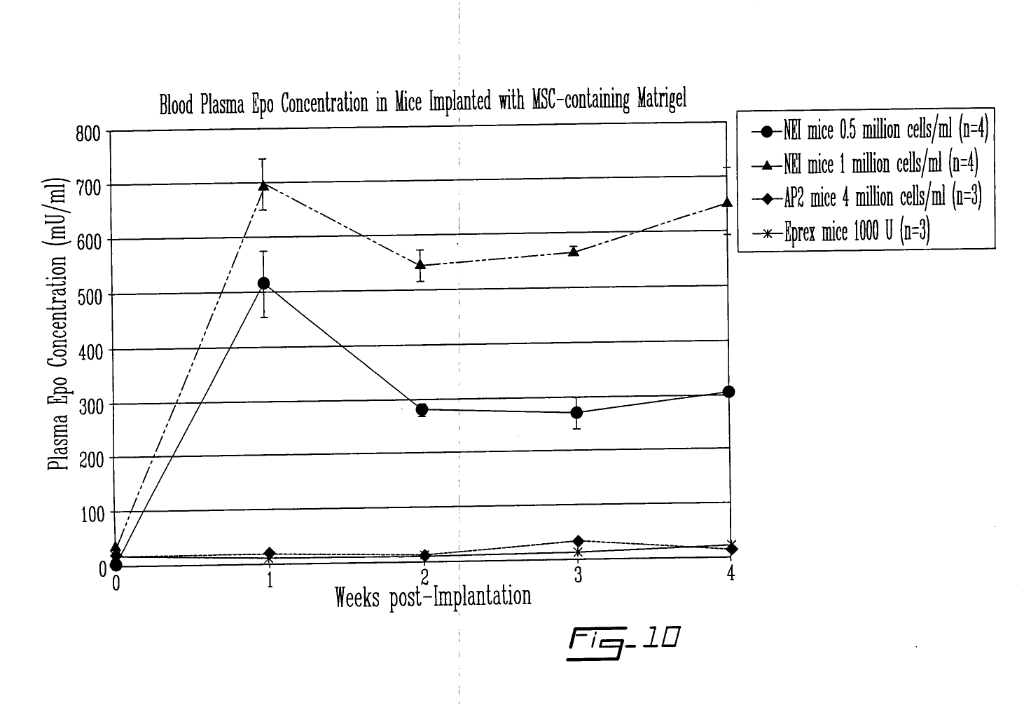

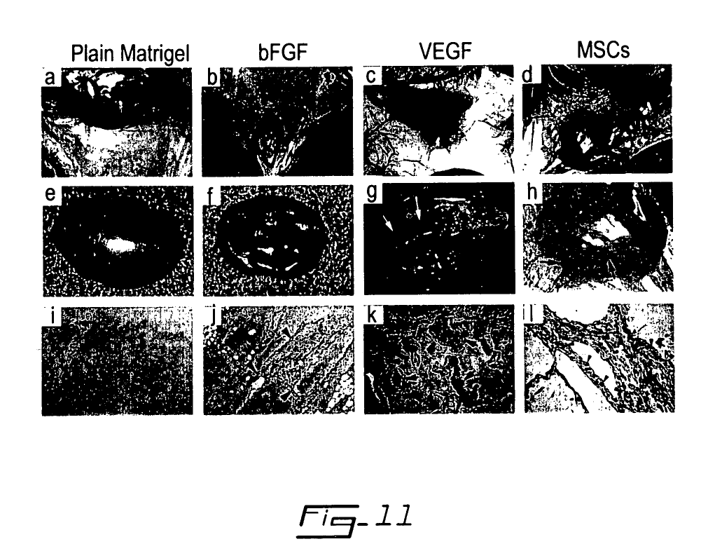

MSCs were harvested from C57BI/6 mice and expanded in culture for 16-20 weeks. MSCs were fibroblast-like in phenotype and no expression of CD31, CD34 or VEGF was detected by immunohistochemical analysis of these cells. The mixed polyclonal population of culture-expanded MSCs was harvested and suspended in Matrigel™ for in vivo implantation. Two weeks after subcutaneous implantation in isogenic C57BI/6 mice, large macroscopic blood vessels grew into Matrigel™ plugs containing MSCs while plain Matrigel™ (negative control) were avascular. The growth of small calibre was noted, tortuous blood vessels in Matrigel™ plugs containing 1000 ng/ml of basic fibroblast growth factor (bFGF) and hemangioma-like structures in Matrigel™ containing 50 ng/ml of murine VEGF 165 (FIGS. 10a to 10). Histological sections confirmed the macroscopic observations. (FIGS. 10m to 10p). The bFGF group characterized by the presence of moderate fibrosis with small disorganized capillaries. In the VEGF group, there were large angiomatous structures lined with thin single endothelial layer with absent to minimal fibrosis. In contrast, the MSC group contained more organized, branching blood vessels ranging from muscular arterioles to small capillaries. The mean vascular density (MVD) in Matrigel™ plugs containing 2.0×10⁶ MSC/ml at 14 days was 41±5 blood vessels (BV)/mm², compared to 21±5, 11±2 and 0.5±0.7 BV/mm² for the VEGF, bFGF and negative control groups, respectively (P<0.001). When the angiogenic response at 4 weeks was assessed, the macroscopic and microscopic differences between the groups were even more evident (FIGS. 11a to 11h). The plain gel plugs continued to be avascular while the bFGF-Matrigel™ plugs showed reduced vascularity with extensive fibrosis. In the VEGF-Matrigel™, there was massive growth of the hemangioma-like structures. In the MSC-Matrigel™ implants, more macroscopic blood vessels developed arranging in a network formation (FIG. 11h). These results were also confirmed by H&E histological staining (FIGS. 11i to 11l). The MVD in gel plugs containing 2.0×10⁶ MSCs/ml at 28 days was 78±9 BV/mm² compared to 11±4, 7±0.8 and 2±0.5 BV/mm² for the VEGF, bFGF and negative control groups, respectively (P<0.001). Comparing results at 14 and 28 days using 2.0×10⁶ MSCs/ml showed a 100% increase in the MVD (P<0.001). The vascular densities associated with different numbers of MSCs (Range 1 to 8×10⁶ MSCs/ml) was compared at 14 days. Results suggested the presence of a dose-response relationship between the number of MSCs/ml and the density of blood vessels. The differences were statistically significant up to 4.0×10⁶ MSCs/ml (P<0.001).

MSCs Stimulate Arteriogenesis

In random sections of the Matrigel™ specimens, the development of arterioles defined by their size (blood vessels>20 μm in diameter) and their structure (blood vessels containing smooth muscle in their wall) were observed. Using Masson's trichrome staining, smooth muscle bundles in the wall of several blood vessels per section occurring only in the MSC-Matrigel™ pellets were observed. None of the sections obtained from VEGF, bFGF or negative control groups contained blood vessels≧20 μm in diameter with smooth muscle in their wall. The density of blood vessels (BV)≧20 μm per mm² was counted and compared results at 14 days with those at 28 days. The number of BV≧20 μm was significantly increased at 14 days in all MSC groups when compared with controls. A further significant 6.25 fold increase (from 1.6±7 to 10±2 BV≧20 μm/mm²) occurred between days 14 and 28 for MSCs, whereas no such phenomena was observed with either bFGF or VEGF.

In Vivo Differentiation of MSC Into Endothelium and Vasculogenesis

In a separate series of experiments, culture-expanded MSCs were retrovirally labelled with LacZ in vitro, Matrigel™-embedded and their subsequent fate in vivo assessed by histochemical analysis with X-gal staining. Histological examination of gel plugs embedded with LacZ⁺MSCs revealed that approximately 20-30% of gene-marked MSCs were associated with the architecture of vascular structures. The other LacZ⁺MSCs were randomly dispersed within the plug with a fibroblast-like histological appearance, many of which were CD31⁺ and VEGF⁺. By far, the majority of cells recruited within the gel plug did not stain blue with X-gal and are of host origin. The majority of host-derived LacZ^{null} cells were part of histologically recognizable vascular structures with little or no inflammatory infiltration by monocytes. LacZ⁺MSCs incorporated in the wall of several blood vessels have been observed. LacZ⁺MSCs in the inner intimal layer where they were flattened and had taken the histological configuration of endothelial cells were also observed. These LacZ⁺MSCs were CD31⁺ and VEGF⁺. These findings are consistent with in vivo phenotypic differentiation of MSCs into endothelium LacZ⁺MSCs in the sub-endothelial layer were also observed, where they were flattened, elongated and aligned circumferentially in the wall of the blood vessel. This was a frequent observation in the wall of large blood vessels. Based on LacZ gene reporter activity, it was found that implanted MSCs contributed to approximately 0.9% of all the new blood vessels. Therefore, the majority of the angiogenic response (˜99.1 %) was from host-derived cells.

The Role of VEGF

Neutralizing anti-murine VEGF antibodies that were mixed with the MSCs in the gel plugs prior to implantation. After two weeks in vivo, there was no visible blood vessels macroscopically and markedly reduced angiogenic response in histological sections, and viable LacZ⁺MSCs were present. The MVD was reduced to 6±2 BV/mm², compared to 37±5 BV/mm² when we used non-specific polyclonal 1 gG antibodies of the same source and class as a control (P<0.001). The use of VEGF neutralizing antibodies was also associated with the disappearance of MSCs expressing CD31. MSCs placed in suspension culture in gel in vitro form spherical colonies. Whereas, the addition of recombinant VEGF 165 (50 ng/ml) induces the formation of clearly recognized capillary tube-like structures and these cells become CD31⁺.

### EXAMPLE VII

### Genetically Engineered Autologous Bone Marrow Stromal Cells in Matrix as a Platform for Systemic Delivery of Erythropoietin

Autologous bone marrow stromal cells are an ideal vehicle for delivery of therapeutic genes. They are easy to harvest, expand in vitro, and genetically engineer with retroviral vectors. In this experiment, the hematopoietic effects of bone marrow stromal cells genetically modified to secrete erythropoietin (Epo) and embedded in subcutaneous matrix implants was examined.

Materials and Methods

Marrow stromal cells (MSCs) were harvested from the bone marrow of C57BI/6 mice and culture expanded. Murine Epo was cloned in the bicistronic retroviral vector CMV˜murine Epo˜IRES˜GFP˜LTR. The resulting construct was stably transfected into GP+E86 packaging cells, consequently generating Epo-GP+E86 cells producing ˜4.0×10⁵ infectious particles per ml, as determined by titer assay on NIH 3T3 cells. MSCs were transduced with these retroparticles once a day for 3 consecutive days. These transduced cells were culture expanded for ˜2-3 months. They were found to secrete ˜17u of Epo/10⁶ cells/24 hours in vitro as revealed by enzyme-linked immunosorbent assay (ELISA). Flow cytometry analysis showed that ˜91% of these cells were expressing GFP. These cells were also transduced with retrovector carrying the LacZ gene. Various numbers of genetically engineered MSCs (0.5×10⁶, 1.0×10⁶, and 8.0×10⁶ cells/ml) were mixed with basement membrane constituent matrix (Matrigel™) and injected subcutaneously into the flank of isogenic mice. Results were compared to mice that received Matrigel™ with either MSCs transduced with a control retrovector (negative control) or with escalating dose of recombinant human Epo (Eprex™).

Results

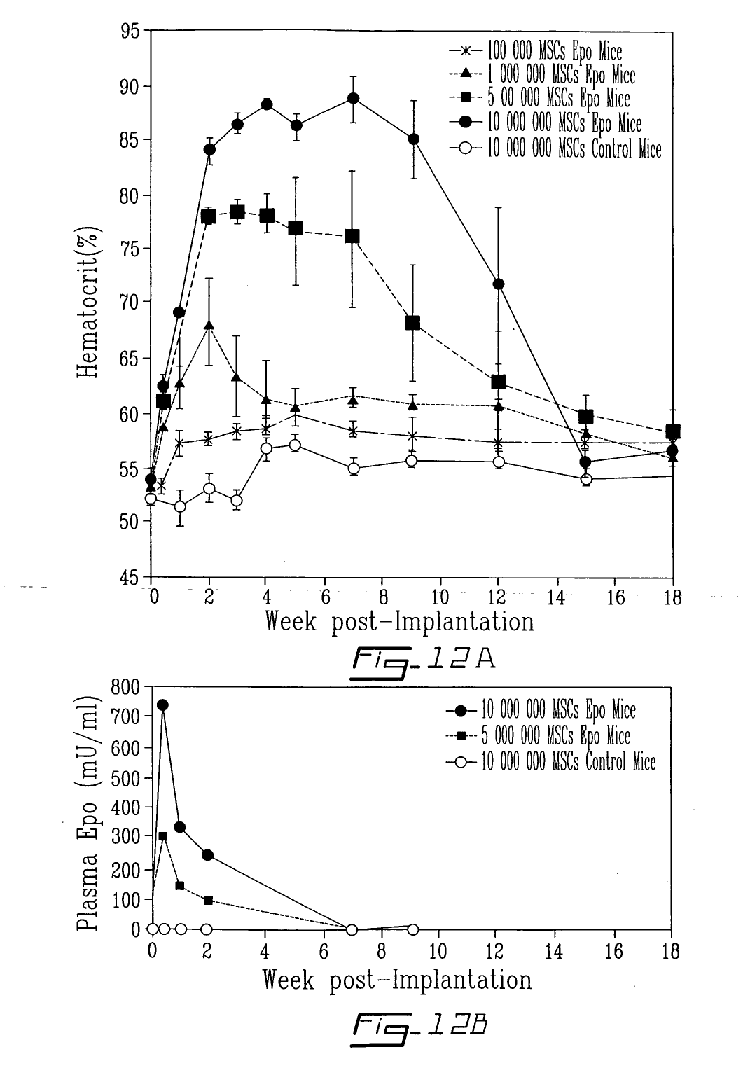

The hematocrit of mice that received Epo secreting MSC rose from a baseline of 53±3% (mean±SD) to 67±1%, 80±2%, and 90±1% with the 0.5×10⁶, 1.0×10⁶, and 8.0×10⁶ cells/ml doses respectively within 2 weeks following implantation and remained constant over the next 2 weeks (FIG. 12). The hematocrit of the negative control group remained at the baseline level (51±3%) over the 4 week period of the study. In the group of mice that received the highest dose of Eprex (1000u in 0.5 ml of Matrigel™), the hematocrit increased from baseline value of 50±2% to 63±2% within 2 weeks and remained constant over the next 2 weeks.

Conclusion

The present findings strongly support that matrix implants containing genetically engineered MSCs can be used for the systemic delivery of erythropoietin or any other therapeutic protein. The ease of implantation and removal makes this approach clinically desirable.

### EXAMPLE VIII

### Marrow Stroma Implant for Erythropoietin Delivery in Normal Mice

Materials and Methods

Cell Culture of Murine Fibroblasts

GP+E86 ecotropic retrovirus-packaging cell line from American Type Culture Collection (ATCC) was cultured in Dulbecco's modified essential medium (DMEM) (Wisent Technologies, St.Bruno, QC) supplemented with 10% heat-inactivated fetal bovine serum (FBS) (Wisent) and 50 Units/ml penicillin, 50 □g/ml streptomycin (Pen/Step) (Wisent). National Institutes of Health (NIH) 3T3 mouse fibroblast cell line, obtained from ATCC, was grown in DMEM with 10% FBS and 50 Units/ml Pen/Step. All cells were maintained in a humidified incubator at 37° C. with 5% C0₂.

Generation of Retroviral Vector and of Virus-Producing Cells

The retroviral plasmid vector pIRES-EGFP was previously generated in our laboratory. This construct comprises a multiple cloning site linked by an internal ribosomal entry site (IRES) to the enhanced green fluorescent protein (EGFP) (Clontech Laboratories, Palo Alto, Calif.). The retroviral vector pEpo-IRES-EGFP (FIG. 1) was synthesized by obtaining the cDNA for mouse Epo by Bam H1 digest of a pBluescript-based construct graciously provided by Jean M. Heard (Institut Pasteur, Paris) and ligating it with a Bam H1 digest of pIRES-EGFP.

For the manufacture of recombinant virus-producing cells, the pEpo-IRES-EGFP construct (50 g) was linearized by Fsp1 digest and co-transfected, utilizing lipofectamine reagent (Gibco-BRL, Gaithesburg, Md.), with 0.5 □g pJ6□Bleo drug resistance plasmid generously given by Richard C. Mulligan (Children's Hospital, Massechusettes), into GP+E86 packaging cells. Stable transfectants were selected by 5-week exposure to 100 □g/ml zeocin (Invitrogen, San Diego, Calif.), thus giving rise to the polyclonal virus-producing cells GP+E86-Epo-IRES-EGFP. GFP expression in cells was assessed by flow cytometry analysis utilizing an Epics XL/MCL Coulter analyzer and gating viable cells based on FSC/SSC profile. A population of Sorted GP+E86-Epo-IRES-EGFP producers was obtained following sorting of GP+E86-Epo-IRES-EGFP cells based on green fluorescence using a Becton Dickinson FACSTAR sorter. The control GP+E86-IRES-EGFP producers were generated in this same manner. Retroparticles from all producers were devoid of replication competent retrovirus as was determined by GFP marker rescue assay employing conditioned supernatants from transduced target cells. GP+E86-LacZ retrovirus producing cells were generated by transinfection of the GP+E86 cell line with filtered retroviral supernatant from 293GPG-LacZ producers (generously provided by R. C. Mulligan, Children's Hospital, Massechusettes) twice per day for 3 consecutive days, in the presence of 6 μg/ml lipofectamine.

Titer Determination of Retrovirus Producers

To assess the titer of GP+E86-Epo-IRES-EGFP and GP+E86-IRES-EGFP producers, NIH 3T3 fibroblasts were seeded at a density of 2 to 4×10⁴ cells per well of 6-well tissue culture plates. The next day, cells were exposed to serial dilutions (0.01 μl to 100 μl) of 0.45 μm filtered retroviral supernatants, in a total volume of 1 ml complete media with 6 μg/ml lipofectamine. Cells from extra test wells were counted and averaged to disclose the baseline cell number at moment of virus addition. Three days later, the percentage of GFP-expressing cells was ascertained by flow cytometry analysis. The titer was calculated using the following equation by considering the virus dilution that yielded 10-40% GFP-positive cells. Titer (infectious particles/ml)=(% GFP-positive cells)×(amount of target cells at start of virus exposure)/(volume of virus in the 1 ml applied to cells). The titer of GP+E86-LacZ virus producers was estimated through X-gal staining of likewise transduced NIH 3T3 cells.

Harvest, Culture, and Transduction of Murine Bone Marrow Stroma

Whole bone marrow was harvested from the femurs and tibias of 18-22 g female C57BI/6 mice (Charles River, Laprairie Co., QC) and plated in DMEM supplemented with 10% FBS and 50 Units/ml Pen/Step. After 4 to 5 days of incubation at 37° C. with 5% CO₂, the nonadherent hematopoietic cells were discarded and the adherent MSCs were gene-modified as follows. Media was removed from MSCs and replaced with 0.45 μm-filtered retroviral supernatant from subconfluent Sorted GP+E86-Epo-IRES-EGFP or control GP+E86-IRES-EGFP producers once per day for six consecutive days, for each of two successive weeks, in the presence of 6 μg/ml lipofectamine. The resulting genetically engineered stromal cells were subsequently expanded for 2-3 months. As additional populations of gene-modified MSCs, Epo-IRES-EGFP modified MSCs as well as control IRES-EGFP MSCs were also transduced with retroparticles from GP+E86-LacZ producers twice per day for three consecutive days with 6 μg/ml lipofectamine, giving rise to LacZ-Epo-IRES-EGFP modified MSCs and LacZ-IRES-EGFP MSCs, respectively. GFP expression in genetically engineered stroma was evaluated by flow cytometry analysis to allow an estimate of the gene transfer efficiency. Beta-galactosidase expression in LacZ gene modified MSCs was determined by X-gal staining. Culture expanded murine MSCs were CD31⁻, CD34⁻, and CD45⁻ in vitro. Supernatant was collected from genetically engineered cells and mouse Epo secretion was assessed by photometric enzyme-linked immunosorbent assay (ELISA) specific for human Epo (Roche Diagnostics, Indianapolis, Ind.).

Southern Blot Analysis

Genomic DNA was isolated from Epo-IRES-EGFP stably transduced primary murine MSCs, as well as from unmodified marrow stroma, utilizing the QlAamp DNA mini kit (Qiagen, Mississauga, ONT). For Southern blot analysis, 10 μg of genomic DNA was digested with EcoRV, separated by electrophoresis in 1% agarose, and transferred to a Hybond-N nylon membrane (Amersham, Oakville, ONT). The probe was prepared by ³²P radiolabeling of the EGFP complete cDNA utilizing a Random Primed DNA Labeling Kit (Roche Diagnostics) and was hybridized with the membrane. The blot was subsequently washed, irradiated, and exposed to Kodak X-Omat film.

Stroma Implantation and Blood Sample Analysis

For the intraperitoneal implantations of “free” cells, Epo-IRES-EGFP modified stromal cells were trypsinized, concentrated by centrifugation, and the various concentrations of 10⁵, 10⁶, 5×10⁶ and 10⁷ cells in 1 ml of serum-free RPMI media (Wisent) were injected into the peritoneum of 4 cohorts of 3 to 4 syngeneic C57BI/6 mice. Control mice (n=5) were implanted with 10⁷ IRES-EGFP modified MSCs. For the subcutaneous implantations of “free” cells, 4×10⁶ Epo-IRES-EGFP modified MSCs were resuspended in 500 □l of RPMI media and injected in the subcutaneous space of each of 5 syngeneic mice. Control mice (n=4) were generated by subcutaneous administration of 4×10⁶ IRES-EGFP MSCs. For the subcutaneous implantations of Matrigel embedded MSCs, 4×10⁶ Epo-IRES-EGFP modified MSCs were resuspended in 50 μl of RPMI media, mixed with 500 μl Matrigel™ (Becton Dickinson) at 4° C. and implanted by subcutaneous injection in the right flank of 3 syngeneic C57BI/6 mice. Matrigel, at body temperature, rapidly acquires a semi-solid form. Control mice (n=4) were implanted with 4×10⁶ Matrigel embedded IRES-EGFP MSCs. In addition, 4×10⁶ LacZ-Epo-IRES-EGFP MSCs mixed in Matrigel were implanted in another 3 mice. Control mice (n=3) received 4×10⁶ LacZ-IRES-EGFP MSCs in Matrigel. For the shorter 4 week study, LacZ-Epo-IRES-EGFP modified MSCs were likewise injected embedded in Matrigel at the various cell doses of 4, 0.5, and 0.25×10⁶ MSCs in each of 4 mice. Control mice (n=4) were equally generated by implantation of 0.5×10⁶ Lac Z-IRES-EGFP MSCs enclosed in Matrigel. As a positive control, 4 mice were administered subcutaneously 1000 Units of human recombinant Epo (Eprex™, Janssen-Ortho Inc., North York ONT) mixed in Matrigel. For the subcutaneous implantation of MSCs embedded in a “human-compatible” bovine type I collagen-based matrix, 4-5×10⁶ Epo-IRES-EGFP modified stromal cells suspended in 150 μl DMEM with 10% FBS were placed on a 1 cm² piece of porous Collagen Matrix (Collagen Matrix, Inc., New Jersey) in a well of a 24 well-plate. The matrix became soaked and 15 minutes later, 800 μl of complete media was added to the well and the MSC-embedded collagen incubated overnight at 37° C. with 5% CO₂. The following day, one MSC-embedded collagen implant was surgically introduced into the subcutaneous space behind the neck of each of 5 syngeneic C57BI/6 mice anesthetized by isoflurane inhalation. Control mice (n=5) were implanted with 4-5×10⁶ IRES-EGFP modified MSCs embedded in Collagen Matrix and 5 additional negative control mice. received the collagen only. Blood samples were collected from the saphenous vein with heparinized micro-hematocrit tubes (Fisher Scientific, Pittsburgh, Pa.) prior to and every ˜1 or more weeks post-implantation. Mice were monitored for up to 10 months. Hematocrit levels and plasma mEpo concentrations were ascertained from blood samples. Specifically, hematocrits were quantitated by standard microhematocrit procedure, and mEpo concentrations in plasma preparations were assessed by ELISA for human Epo (Roche Diagnostics).

Matrigel Implant Removal and Processing

At 4 weeks post-implantation, mice implanted with LacZ gene-modified. MSCs (i.e. LacZ-Epo-IRES-EGFP MSCs and LacZ-IRES-EGFP MSCs) embedded in Matrigel were sacrificed and their systemic circulation flushed through the left ventricle with 15 ml of 4° C phosphate buffered solution (PBS) and then with 15 ml of 4° C. 2% paraformaldehyde (PFA).

Matrigel implants were recovered and immersed in 2% PFA at 4° C. for 24 hours and in X-gal solution (5 mM K₃Fe(CN)₆, 5 mM K₄Fe(CN)₆.3H₂O, 0.01% sodium deoxycholate, 2 mM MgCl₂, 1 mM EGTA, and 1 mg/ml X-gal in PBS with 0.02% NP40) for 16 hours. Samples were then fixed with 10% formalin, embedded in paraffin and sections of 3-4 μm were prepared. For immunohistochemical staining, specimens were deparaffinized in toluene and rehydrated. Endogenous peroxidase was blocked using 3% hydrogen peroxide followed by incubation with 5% bovine serum albumin with 5% goat serum or 5% donkey serum in PBS for 30 minutes. Sections were placed at 37° C. with primary antibodies (polyclonal goat ant-mouse CD31 at 1:100), followed by biotin-conjugated secondary antibodies (donkey anti-goat IgG from Santa Cruz at 1:100, or goat anti-rabbit at 1:200 from BD Pharmingen), washed, and treated with avidin-peroxidase (ABC Elite kit, Vector Laboratories) for 30 minutes. DAB substrate (Vector Laboratories) was used for reaction development. Sections were counterstained with hematoxylin and eosin, visualized with an Olympus BX60 microscope, and digital images retrieved on a computer equipped with Image Pro software (Media Cybernetics).

Results

Marrow Stromal Cells (MSCs) are postnatal progenitor cells that can be easily cultured ex vivo to large amounts. This feature is attractive for cell therapy applications where genetically engineered MSCs could serve as an autologous cellular vehicle for the delivery of therapeutic proteins. The usefulness of MSCs in transgenic cell therapy will rely upon their potential to engraft in non-myeloablated, immunocompetent recipients. Further, the ability to deliver MSCs subcutaneously—as opposed to intravenous or intraperitoneal infusions—would enhance safety by providing an easily accessible, and retrievable, artificial subcutaneous implant in a clinical setting. To test this hypothesis, MSCs were retrovirally-engineered to secrete mouse erythropoietin (Epo) and their effect was ascertained in non-myeloablated syngeneic mice. Epo-secreting MSCs when administered as “free” cells by subcutaneous or intraperitoneal injection, at the same cell dose, led to a significant—yet temporary—hematocrit increase to over 70% for 55±13 days. In contrast, in mice implanted subcutaneously with Matrigel™-embedded MSCs, the hematocrit persisted at levels >80% for over 110 days in 4 of 6 mice (p<0.05 logrank). Moreover, Epo-secreting MSCs mixed in Matrigel elicited and directly participated in blood vessel formation de novo reflecting their mesenchymal plasticity. MSCs embedded in human-compatible bovine collagen matrix also led to a hematocrit >70% for 75±8.9 days. In conclusion, matrix-embedded MSCs will spontaneously form a neovascularized organoid that supports the release of a soluble plasma protein directly into the bloodstream for a sustained pharmacological effect in non-myeloablated recipients.

Titer of Retrovirus Producers

To determine gene transfer efficiency and transgene expression in stably transfected retroviral producer cells, flow cytometry analysis for GFP expression was performed. The proportion of GFP positive cells in the polyclonal producer populations GP+E86-Epo-IRES-EGFP, and GP+E86-Epo-IRES-EGFP Sorted based on green fluorescence, was 34% and 97%, respectively. To evaluate the quantity of infections particles released by these producers, a titration assay using their retroviral supernatant was conducted and the viral titers obtained were ˜2.4×10⁵ and ˜4.0×10⁵ infections particles per ml, respectively. The percentage of LacZ positive cells in the GP+E86-LacZ viral producer cell population was >95% and the viral titer of these cells was ˜1.1×10⁵ infections particles per ml.

Retrovector Expression and mEpo Secretion by Gene-Modified Marrow Stroma

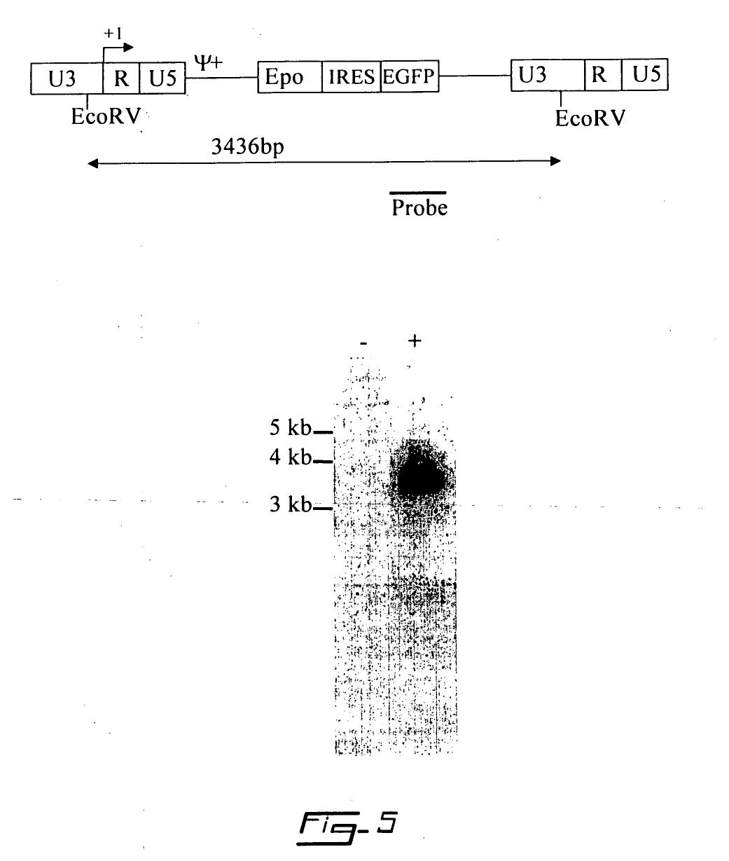

To determine the molecular genetic stability of the Epo-IRES-EGFP retroviral construct, proviral DNA in the genome of polyclonal retrovirally-transduced MSCs was analyzed by Southern blot. A probe complementary to the GFP reporter allowed the detection of a DNA band consistent with the 3436 bp fragment anticipated from EcoRV digest of integrated unrearranged Epo-IRES-EGFP proviral DNA (FIG. 5). No subgenomic or rearranged retrovector integrant was detected.

Retrovector expression in genetically engineered murine MSCs was confirmed by flow cytrometry analysis for GFP expression. The proportion of Epo-IRES-EGFP modified MSCs expressing GFP was 91%. To establish that murine MSCs transduced with Epo-IRES-EGFP secrete mEpo in vitro, and quantitate the level, supernatant collected from these cells was analyzed by ELISA for human Epo. The Epo-IRES-EGFP modified MSC population was analyzed and found to secrete 17 Units of Epo per 10⁶ cells per 24 hours, respectively. The percentage of LacZ positive cells in the LacZ-Epo-IRES-EGFP modified MSC population was >90%. LacZ-Epo-IRES-EGFP modified stroma was noted to secrete 17 Units of Epo per 10⁶ cells per 24 hours. There was no Epo detected in the supernatant collected from control IRES-EGFP transduced MSCs and LacZ-IRES-EGFP MSCs.

Intraperitoneal Implantation of Epo-Secreting MSCs

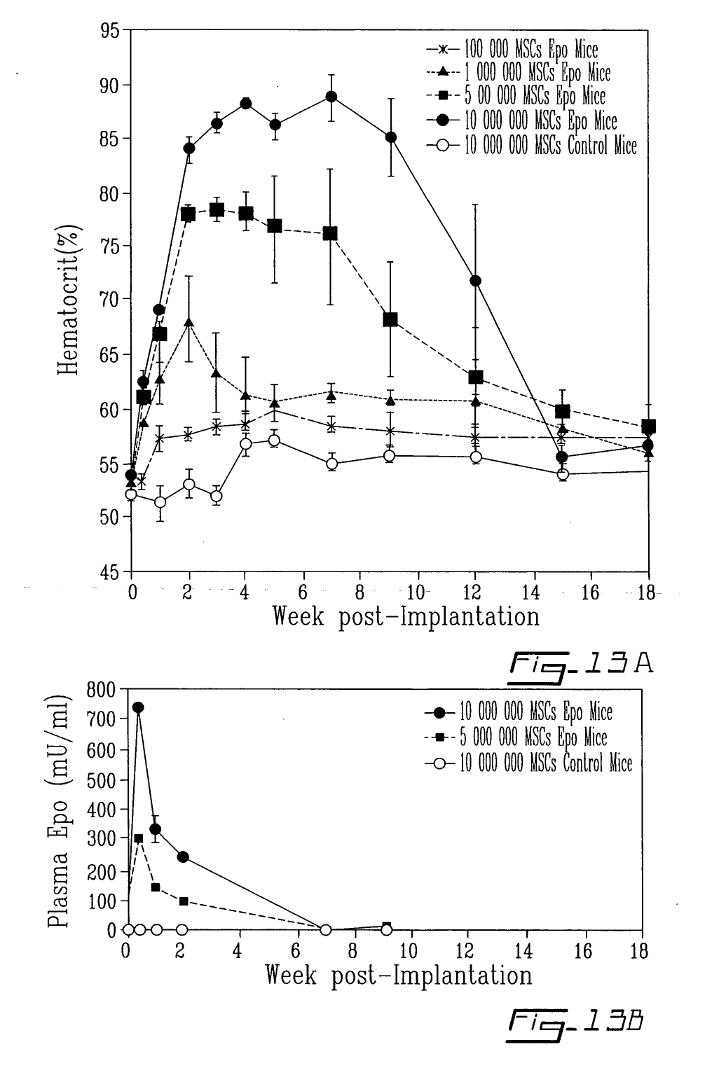

We determined if mEpo secretion from Epo-IRES-EGFP transduced MSCs implanted by intraperitoneal injection in non-myeloablated, immunocompetent mice can lead to a measurable effect on hematocrit. We also established if there is a dose-response relationship between the number of Epo-IRES-EGFP modified stromal cells injected and the resulting hematocrit. Cohorts of mice were implanted with either 10⁵, 10⁶, 5×10⁶ or 10⁷ of Epo-IRES-EGFP engineered MSCs. Peripheral blood was collected and hematocrit and plasma Epo concentration measured over time as shown in FIG. 13. As illustrated in FIG. 13A, the hematocrit of mice that received 10⁵ mEpo-secreting stromal cells rose to a peak value of 60±1.1% at 5 weeks post-implantation. In mice injected with 10⁶ Epo-IRES-EGFP transduced MSCs, blood hematocrit rose to maximum of 68±3.8% at 2 weeks following implantation and then quickly declined to a steady ˜61% observed until week 12. The recipients of 5×10⁶ mEpo secreting MSCs had an increase in hematocrit that attained a value of ˜78% at 2 weeks post-implantation, remaining above 75% until 7 weeks following stroma administration. The hematocrit of mice implanted with 10⁷ of these gene-modified MSCs attained the highest level at 4 weeks (˜88%), thenceforth persisting at ˜85% or greater up to week 9 and over 70% up to week 12. A parallel group of mice received 10⁷ IRES-EGFP transduced MSCs. These control mice maintained hematocrit levels ranging between 51 and 57% throughout this study (FIG. 13A). A tight correlation was revealed between the number of i.p. implanted Epo-secreting MSCs and the resulting peak in the hematocrit (r=0.97).

To quantify the plasma concentration of mouse Epo in mice administered Epo-IRES-EGFP engineered marrow stroma, plasma Epo levels were measured by human Epo ELISA. As done by others in the field we utilized ELISA kits for detection of human Epo to detect mouse Epo. Though affinity for mEpo is poor, it remains the standard in the field and serves as a basis for comparison. Therefore, our measured plasma mEpo concentrations are likely underestimated due to levels below the threshold of detectability of this kit. Mice that received by intraperitoneal injection 10⁷ and 5×10⁶ Epo-IRES-EGFP engineered MSCs secretrig in vitro 17 Units of Epo per 10⁶cells per 24 hours, exhibited a rise in plasma Epo levels to 740±20 and 298±25 mUnits/ml, respectively, at 3 days post-implantation (FIG. 13B), which declined proportionally by over 50% to 333±60 and 141±15 mUnits/mi, respectively, at 1 week, and by over 65% to 255±15 and 96±18 mUnits/ml, respectively, at 2 weeks. The concentration of Epo detected in the plasma of these mice at 7 weeks or greater post-implantation was under 20 mUnits/ml.

Subcutaneous Implantation of Matrigel-Embedded, Epo-Secreting MSCs

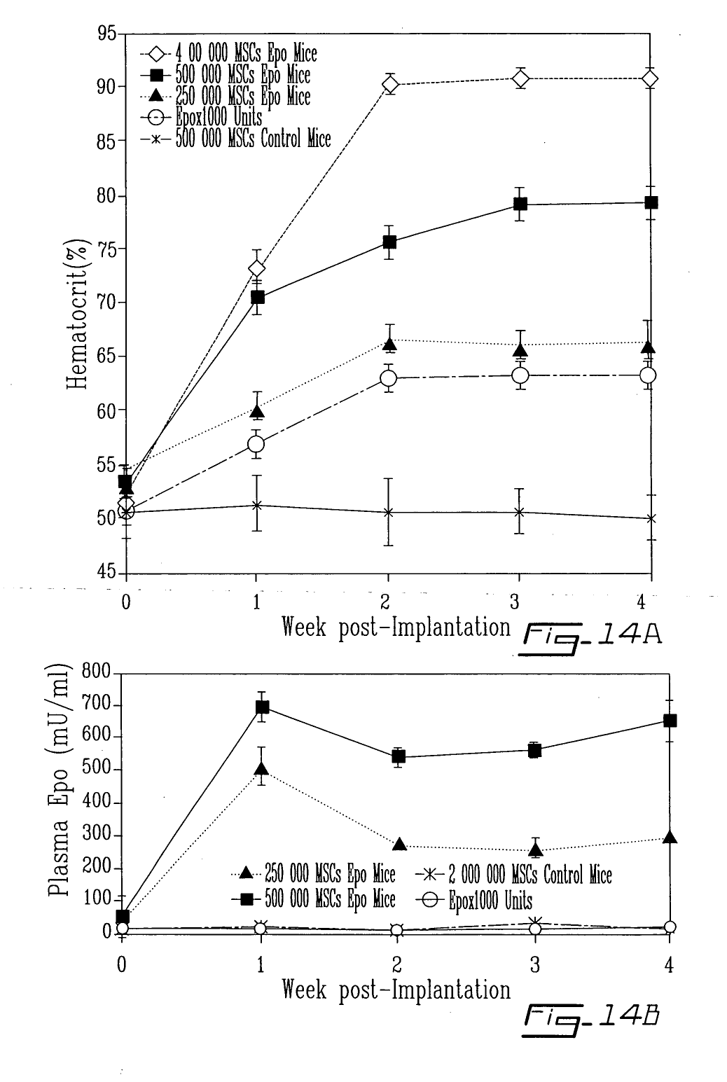

As an alternative delivery route, we tested whether mEpo engineered MSCs implanted in the subcutaneous space display the same pharmacological features as intraperitoneal delivery. We also conducted subcutaneous implantations of gene-modified MSCs pre-mixed in Matrigel. Peripheral blood was collected and hematocrit and plasma Epo concentration measured over time as represented in FIG. 14. To first ascertain if there is a correlation between the number of Epo-secreting MSCs mixed in Matrigel and the consequent rise in hematocrit during the first four weeks post-implantation, groups of C57BI/6 mice were injected subcutaneously with 4, 0.5, and 0.2×5×10⁶ LacZ-Epo-IRES-EGFP modified MSCs per mouse. The hematocrit of these mice increased from a baseline of 53±3% (mean±SD) to 90±1%, 80±2%, and 67±1%, respectively, within 2 weeks following implantation, as shown in FIG. 14A. The Epo-secreting MSC dose and resulting hematocrit correlated strongly (r=0.90). The hematocrit of the negative control group generated by implantation of Matrigel-embedded LacZ-IRES-EGFP MSCs maintained the baseline values (51±3%) over the 4 week period of the experiment. As a comparison, we determined the effect of Matrigel admixed with recombinant human Epo (rhuEpo) only. We found that in mice implanted with Matrigel/rhuEpo (100 Units in 0.5 ml of Matrigel, or ˜40,000 Units/kg), the hematocrit increased from 50±2% to 63±2% within 2 weeks and was thereafter sustained for the subsequent 2 weeks. The pattern in the change of hematocrit over time with rhuEpo was similar to that achieved when mice received the lowest tested dose of 0.25×10⁶ Epo-secreting MSCs (FIG. 14A).

To determine the concentration of mouse Epo in blood plasma of mice subcutaneously injected with Epo-secreting MSCs embedded in Matrigel, human Epo ELISA was performed. In mice implanted with these Matrigel embedded MSCs, the plasma Epo concentration increased from <30 mU/ml prior to implantation to ˜510, 280, and 270 mU/ml with 0.25×10⁶ LacZ-Epo-IRES-EGFP modified MSCs at 1, 2, and 3-weeks post-implantation respectively (FIG. 14B). At these time points, 0.5×10⁶ LacZ-Epo-IRES-EGFP MSCs led to plasma Epo levels of ˜700, 540, and 570 mU/ml. Values observed at 4 weeks were similar to those at 2 and 3 weeks following implantation. In mice implanted with Matrigel mixed with LacZ-IRES-EGFP MSCs or rhuEpo, the concentration of Epo detected was <35 mU/ml. Unlike the change in hematocrit observed over time with rhuEpo, plasma Epo levels were not altered (FIG. 14).

In Vivo Endothelial Differentiation of Matrigel-Embedded Epo-Secreting MSCs

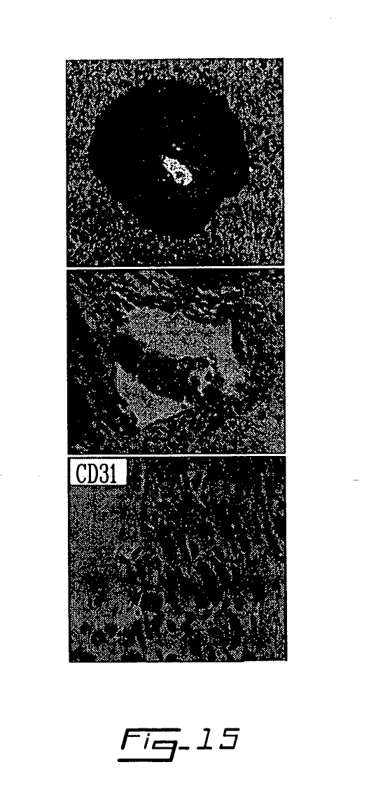

To study the in vivo fate of Epo-secreting MSCs mixed in Matrigel, these cells were gene-modified to also express β-galactosidase (LacZ-Epo-IRES-EGFP MSCs). X-gal histochemical analysis of surgically excised implants was subsequently performed at 4 weeks post-implantation. Macroscopic examination revealed the occurrence of blood vessels within. MSC-containing Matrigel implants (FIG. 15A). Sections of the implant were prepared to show transgene expressing cells based on LacZ gene reporter activity. By X-gal staining, we detected the β-galactosidase expressing Epo-producing MSCs randomly dispersed within the implant with a fibroblast-like histological appearance but additionally, as shown in FIG. 15B, incorporated in the wall of blood vessels. As evidenced in FIG. 15C, these cells had adopted the histological configuration of endothelial cells and had become CD31⁺, results consistent with the in vivo phenotypic differentiation of MSCs into endothelium.

Long-Term Hematocrit Following Subcutaneous Implantation of Epo-Secreting MSCs in Matrices

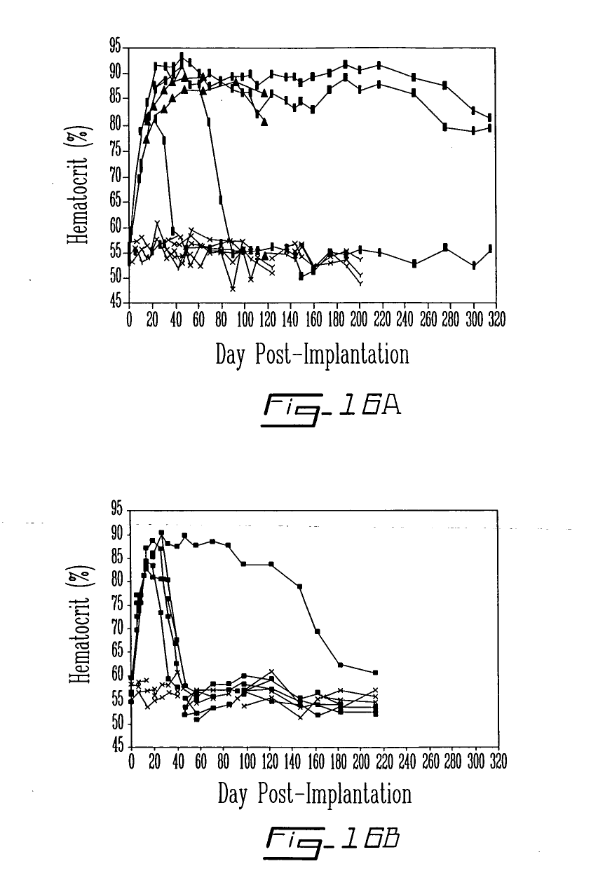

In order to assess if providing MSCs with an artificial microenvironment is of importance for sustained pharmacological production of Epo, we compared the long-term impact on hematocrit of MSCs delivered freely in the subcutaneous space with MSCs mixed in Matrigel. As shown in FIG. 16A, in C57BI/6 mice implanted with 4×10⁶ Matrigel-embedded Epo-IRES-EGFP MSCs, the hematocrit increased from a basal 55±0.7% (Mean±SEM) to 82±1.2 % at 17 days post-implantation and persisted at levels of 80-90% until day 70 in one mouse, and for over 300 days in the other two recipient mice. Control mice were generated by implantation with 4×10⁶ Matrigel-embedded IRES-EGFP MSCs and demonstrated a consistent Hct of ˜55% over time (FIG. 16A). In a seperate experiment where 4×10⁶ LacZ-Epo-IRES-EGFP MSCs mixed in Matrigel were injected in another 3 mice, 2 of 3 recipient animals showed Hcts above 80% from day 22 to past day 118 post-implantation (FIG. 16A). Control mice (n=3) which received 4×10⁶ LacZ-IRES-EGFP MSCs in Matrigel maintained an Hct of ˜55% (FIG. 16A). In contrast, for the same number of Epo-IRES-EGFP MSCs in the absence of Matrigel, the Hct rose from a basal 56±0.3% (Mean±SEM) before implantation to a peak level of 85±0.9% at 14 days post-implantation which persisted for an additional 14 days, and thereafter declined rapidly in 4 of 5 mice and attained basal values at ˜50 days (FIG. 16B). One mouse maintained hematocrit values above 70% ˜150 days. Control mice implanted with 4×10⁶ MSCs engineered with an Epo-less retrovector demonstrated stable Hct levels of ˜55% (FIG. 16B). A significant difference on long-term effect on Hct was noted between the Matrigel embedded Epo-secreting MSCs when compared to the unembedded cells (p=0.0348 LogRank).

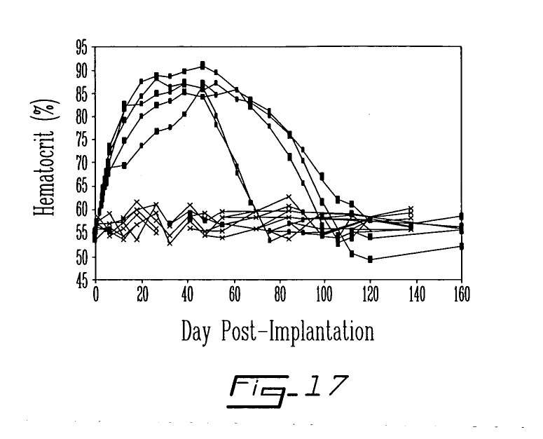

Matrigel is immunologically incompatible with non-murine species. Amongst the many components of Matrigel, collagen figures prominently and may play an important role as part of the artificial microenvironment provided by Matrigel to MSCs. We hypothesized that a human-compatible type I bovine-derived collagen pharmaceutical-grade product could serve as a substitute for Matrigel, thereby offering clues toward clinically-feasible application of this strategy. As shown in FIG. 17, 4-5×10⁶ collagen-embedded Epo-IRES-EGFP MSCs led to a significant increase in Hct compared with controls (FIG. 17). Specifically, in mice (n=5) implanted with Collagen Matrix embedded Epo-secreting MSCs, the Hct increased from 55±0.3% to a peak level of 82±2.4 % at 20 days and thereafter gradually decreased. A significant difference in Hct was observed between mice implanted with Collagen Matrix embedded Epo-secreting MSCs and control mice (P<0.001) (FIG. 17). The effect on Hct was lost in all mice by 120 days post-implantation. We noted that the decline in Hct was concurrent with the physical disappearance of the implant that was palpable in the first weeks and gradually resorbed. When comparing the long-term effect on Hct, all mice implanted with Collagen Matrix embedded Epo-secreting MSCs sustained a Hct above 70% for 75±8.9 days whereas in most mice (4 of 5) which received unembedded cells, this level lasted 32±1.5 days.

### EXAMPLE IX

### Marrow Stromal Cells Retrovirally Engineered to Secrete Interleukin-2 for Cellular Immunotherapy of Cancer

Tumor-localized expression of immunostimulatory cytokines can result in antitumor immune responses in various animal models of cancer. Genetically-engineered marrow stromal cells (MSCs) represent an ideal cellular vehicle for local delivery of anti-cancer proteins because they can be readily collected in patients of all age groups, they can be expanded ex vivo for more than 50 population doublings without signs of differentiation or senescence and they can be easily gene modified with replication-defective retrovectors. We investigated whether MSCs could serve as a novel autologous delivery vehicle of anti-cancer immunostimulatory cytokines, specifically interleukin-2 (IL-2), to the tumor's environment in B16 melanoma. Primary MSCs were isolated from C57BI/6 mice and expanded in vitro. MSCs were gene modified using ecotropic retrovectors to express a bicistronic construct encoding the murine interleukin-2 (mIL-2) cDNA and the reporter GFP (MSC-IL2), or only GFP (MSC-GFP). Single clones were isolated and stable transgene integration was confirmed by Southern blot analysis. Four MSC-derived clones secreting respectively 340 ng (MSC-IL2-high), 211 ng, ˜160 ng and 130 ng of mIL-2/24 h/10⁶ MSCs were selected. The level of mIL-2 secreted correlated directly with the number of integrated retrovector copies as determined by integration site analysis. In a first set of experiments, 10⁵ B16-F0 cells were mixed in vitro with 10⁶ MSC-IL2-high and injected subcutaneously in syngeneic C57BI/6 mice (n=7). Tumor growth was monitored and compared to control groups consisting of 10⁵ B16-F0 mixed with 10⁶ MSC-GFP cells, or 10⁵ B16-F0 alone (n=7 per group). All mice injected with B16 alone or injected with B16+MSC-GFP developed palpable tumors by 10 days post-injection. In contrast, it took 35 days before all mice injected with B16+10⁶ MSC-IL2-high developed palpable tumors (p<0.0001 by Log rank). We evaluated the dose/effect of IL-2-producing MSCs in delaying tumor growth by mixing 10⁵ B16-F0 cells with a range of MSC-IL2-high. We observed a direct correlation between the level of IL-2 secreted by MSCs and the delay in tumor growth. This anti-tumor dose/effect was also observed using distinct MSC-IL2 clones (R²=0.93). The in vivo immune infiltration mediated by MSC-IL2 was characterized by flow cytometry. Early lymphocytic infiltration (day 5) in the control tumors consisted mainly of CD4+ T cells and natural killer cells (32% and 18% respectively), while MSC-IL2 embedded tumors were robustly infiltrated with natural killer cells (65%) and fewer (10%) CD4+ T cells. The pattern of the immune infiltrate was similar at day 10 (all p values <0.01 between test and control groups). Histological analysis of tumor sections revealed that engineered MSCs are gradually lost over a period of 12 days following injection, suggesting that the observed decline in anti-tumor effect is likely due to loss of MSC-IL2 over time. In conclusion, MSCs represent an abundant source of autologous cells easily accessible with little manipulation and IL2-transduced clonal populations are rapidly expandable in vitro. Our data support the hypothesis that MSCs can be implanted in tumor environment and that paracrine delivery of cytokines such as IL-2 leads to an immune anti-cancer effect.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. An isolated transgenic bone marrow stromal cell for in vivo delivery of a protein of interest into a patient, wherein said stromal cell is genetically-engineered with an expression vector comprising:
a suitable promoter;
an internal ribosome entry site (IRES);
a first nucleotidic sequence encoding a suitable selectable marker;
a second nucleotidic sequence encoding for said protein of interest; and
a retroviral long terminal repeat (LTR) sequence flanking at 5′ and/or 3′ ends of said vector;
wherein said first and second nucleotidic sequences are operably linked one to the other separated by said IRES, and said selectable marker indicating transgenic cells capable of expressing said second nucleotidic sequence.

2. The stromal cell of claim 1, wherein said patient is an immunocompetent patient.

3. The stromal cell of claim 1, wherein said expression vector is a bicistronic retroviral vector.

4. The stromal cell of claim 1, wherein said expression vector is DNA or RNA.

5. The stromal cell of claim 1, wherein said selectable marker is selected from the group consisting of drug resistance, enhanced green fluorescent protein (EGFP), and β-galactosidase.

6. The stromal cell of claim 1, wherein said protein of interest is endogenous or exogenous.

7. The stromal cell of claim 1, wherein said protein of interest is selected from the group consisting of cytokine, interleukin, growth hormones, hormones, blood factors, marker proteins, immunoglobulins, antigens, releasing hormone, anticancer product, antitumor product, antiviral product, antiretroviral product, an antisense, an antiangiogenic product, an angiogenic product, and a replication inhibitor.

8. The stromal cell of claim 1, wherein said protein of interest is erythropoietin, an analog or a fragment thereof.

9. The stromal cell of claim 1, wherein said promoter comprises a retroviral or synthetic promoter.

10. The stromal cell of claim 1, wherein said patient is a human or an animal.

11. A method of preparing a transgenic bone marrow stromal cell for delivery of a protein of interest into a patient comprising the steps of:
c) providing an isolated stromal cell and culturing said cell in vitro; and
d) introducing an expression vector into said isolated marrow stromal cell, wherein said expression vector comprises:
a suitable promoter;
an internal ribosome entry site (IRES);
a first nucleotidic sequence encoding a suitable selectable marker;
a second nucleotidic sequence encoding for said protein of interest; and
a retroviral long terminal repeat (LTR) sequence flanking at 5′ and/or 3′ ends of said vector;
wherein said first and second nucleotidic sequences are operably linked to and separated by said IRES, and said selectable marker indicating transgenic cells capable of expressing said second nucleotidic sequence.

12. The method of claim 11, wherein said patient is an immunocompetent patient.

13. The method of claim 11, wherein said expression vector is a bicistronic retroviral vector.

14. The method of claim 11, wherein said expression vector is DNA or RNA.

15. The method of claim 11, wherein said selectable marker is selected from the group consisting of drug resistance, enhanced green fluorescent protein (EGFP), and β-galactosidase.

16. The method of claim 11, wherein said protein of interest is endogenous or exogenous.

17. The method of claim 11, wherein said protein of interest is selected from the group consisting of cytokine, interleukin, growth hormones, hormones, blood factors, marker proteins, immunoglobulins, antigens, releasing hormone, anticancer product, antitumor product, antiviral product, antiretroviral product, an antisense, an antiangiogenic product, an angiogenic product, and a replication inhibitor.

18. The method of claim 11, wherein said protein of interest is erythropoietin, an analog or a fragment thereof.

19. The method of claim 11, wherein said promoter comprises a CMV promoter.

20. The method of claim 11, wherein said patient is a human or an animal.

21. A method of introducing and expressing a foreign nucleotidic sequence into a patient comprising the step of:
a) providing an isolated bone marrow stromal cell and culturing said cell in vitro;
b) introducing an expression vector into said isolated stromal cell, wherein said expression vector comprises:
a suitable promoter;
an internal ribosome entry site (IRES);
a first nucleotidic sequence encoding a suitable selectable marker;
a second nucleotidic sequence encoding for said protein of interest; and
a retroviral long terminal repeat (LTR) sequence flanking at 5′ and/or 3′ ends of said vector;
wherein said first and second nucleotidic sequences are operably linked to and separated by said IRES, and said selectable marker indicating transgenic cells capable of expressing said second nucleotidic sequence; and
c) implanting said trangenic stromal cell of step b) into an a patient, wherein said implanted cells produce and secrete the protein of interest.

22. The stromal cell of claim 21, wherein said patient is an immunocompetent patient.

23. The method of claim 21, wherein said expression vector is a bicistronic retroviral vector.

24. The method of claim 21, wherein said expression vector DNA or RNA.

25. The method of claim 21, wherein said selectable marker is selected from the group consisting of drug resistance, enhanced green fluorescent protein (EGFP), and β-galactosidase.

26. The method of claim 21, wherein said protein of interest is endogenous or exogenous.

27. The method of claim 21, wherein said protein of interest is selected from the group consisting of cytokine, interleukin, growth hormones, hormones, blood factors, marker proteins, immunoglobulins, antigens, releasing hormone, anticancer product, antitumor product, antiviral product, antiretroviral product, an antisense, an antiangiogenic product, an angiogenic product, and a replication inhibitor.

28. The method of claim 21, wherein said protein of interest is erythropoietin, an analog or a fragment thereof.

29. The method of claim 21, wherein said promoter comprises a retroviral or synthetic promoter.

30. The stromal cell of claim 21, wherein said patient is a human or an animal.

31. An implant containing cells for modulating tissue synthesis, tissue repair and/or endogenous product synthesis in a patient, said implant comprising a matrix containing viable non genetically manipulated bone marrow stromal cells or bone marrow stromal cells as defined in claim 1, dispersed therein.

32. The implant of claim 31, wherein said patient is a human or an animal.

33. The implant of claim 31, wherein said matrix is selected from the group consisting of chitosan, glycosaminoglycan, chitin, ubiquitin, elastin, polyethylen glycol, polyethylen oxide, vimentin, fibronectin, collagen, derivatives thereof, and combinations thereof.

34. The implant of claim 31, wherein said modulation is revitalization, stimulation, induction, or inhibition of tissues synthesis, tissue repair and/or endogenous product synthesis.

35. The implant of claim 31 or 34, wherein said tissue synthesis is angiogenesis.

36. The implant of claim 31 or 34, wherein said product is selected from the group consisting of lipids, peptides, hormones, glucides, and cytokines.

37. The implant of claim 31, wherein said stromal cells are further genetically engineered.

38. The implant of claim 37, wherein said genetically engineered cells are transgenic cells.

39. The implant of claim 38, wherein said transgenic cells are genetically transformed with an expression vector comprising:
a suitable promoter;
an internal ribosome entry site (IRES);
a first nucleotidic sequence encoding a suitable selectable marker; and/or
a nucleotidic sequence of interest encoding for said protein of interest; and
a retroviral long terminal repeat (LTR) sequence flanking at 5′ and/or 3′ ends of said vector;
wherein said first and nucleotidic sequences of interest are operably linked one to the other separated by said IRES, and said selectable marker indicating transgenic cells capable of expressing said nucleotidic sequence of interest.

40. The implant of claim 39, wherein said expression vector is a bicistronic retroviral vector.

41. The implant of claim 39, wherein said expression vector is DNA or RNA.

42. The implant of claim 39, wherein said selectable marker is selected from the group consisting of drug resistance cytidine deaminase (CD), enhanced green fluorescent protein (EGFP), and β-galactosidase.

43. The implant of claim 39, wherein said protein of interest is endogenous or exogenous.

44. The implant of claim 39, wherein said protein of interest is selected from the group consisting of cytokine, interleukin, growth hormones, hormones, blood factors, marker proteins, immunoglobulins, antigens, releasing hormone, anticancer product, antitumor product, antiviral product, antiretroviral product, an antisense, an antiangiogenic product, an angiogenic product, and a replication inhibitor.

45. The implant of claim 39, wherein said protein of interest is erythropoietin, an analog or a fragment thereof.

46. The implant of claim 39, wherein said promoter comprises a retroviral or synthetic promoter.

47. A method of modulating tissue synthesis, tissue repair and/or endogenous product synthesis in a patient comprising the steps of:
a) providing an isolated bone marrow stromal cell and culturing said cell in vitro;
b) colonizing a biocompatible matrix with said stromal cells of step a); and
c) implanting said colonized matrix of step b) into a patent, wherein said implanted colonized matrix allows for colonizing stromal cells to modulate tissue synthesis, tissue repair and/or endogenous product synthesis in said patient.

48. The method of claim 47, wherein said matrix is selected from the group consisting of chitosan,. glycosaminoglycan, chitin, ubiquitin, elastin, polyethylen glycol, polyethylen oxide, vimentin, fibronectin, collagen, derivatives thereof, and combination thereof.

49. The method of claim 47, wherein said modulation is revitalization, stimulation, induction, or inhibition of tissues synthesis, tissue repair and/or endogenous product synthesis.

50. The method of claim 47 or 49, wherein said tissue synthesis is angiogenesis.

51. The method of claim 47 or 49, wherein said product is selected from the group consisting of lipids, peptides, hormones, glucides, and cytokines.

52. The method of claim 47, wherein said stromal cells are further genetically engineered.

53. The method of claim 52, wherein said genetically engineered cells are transgenic cells.

54. The method of claim 53, wherein said transgenic cells are genetically transformed with an expression vector comprising:
a suitable promoter;
an internal ribosome entry site (IRES);
a first nucleotidic sequence encoding a suitable selectable marker; and/or
a nucleotidic sequence of interest encoding for said protein of interest; and
a retroviral long terminal repeat (LTR) sequence flanking at 5′ and/or 3′ ends of said vector;
wherein said first and nucleotidic sequences of interest are operably linked one to the other separated by said IRES, and said selectable marker indicating transgenic cells capable of expressing said nucleotidic sequence of interest.

55. The method of claim 54, wherein said expression vector is a bicistronic retroviral vector.

56. The method of claim 47, wherein said patient is a human or an animal.

57. The method of claim 54, wherein said expression vector is DNA or RNA.

58. The method of claim 54, wherein said selectable marker is selected from the group consisting of drug resistance, enhanced green fluorescent protein (EGFP), and β-galactosidase.

59. The method of claim 54, wherein said protein of interest is endogenous or exogenous.

60. The method of claim 54, wherein said protein of interest is selected from the group consisting of cytokine, interleukin, growth hormones, hormones, blood factors, marker proteins, immunoglobulins, antigens, releasing hormone, anticancer product, antitumor product, antiviral product, antiretroviral product, an antisense, an antiangiogenic product, an angiogenic product, and a replication inhibitor.

61. The method of claim 54 herein said protein of interest is erythropoietin, an analog or a fragment thereof.

62. The method of claim 54, wherein said promoter comprises a retroviral or synthetic promoter.
